(19) United States
(12) Patent Application Publication
(10) Pub. No.: US 2018/0228775 A1
(43) Pub. Date: Aug. 16, 2018

(54) METHODS OF TREATING CANCER

(71) Applicant: Dana-Farber Cancer Institute, Inc., Boston, MA (US)

(72) Inventors: Girija GOYAL, Cambridge, MA (US); Glenn DRANOFF, Sudbury, MA (US)

(21) Appl. No.: 15/509,566

(22) PCT Filed: Sep. 8, 2015
(86) PCT No.: PCT/US2015/048925

§ 371 Date: Mar. 8, 2017

Related U.S. Application Data
(60) Provisional application No. 62/055,234, filed on Sep. 25, 2014.
(60) Provisional application No. 62/047,467, filed on Sep. 8, 2014.

(51) Int. Cl. A61K 31/426; C07K 16/28; A61K 35/13; A61K 35/17; A61K 38/19; A61K 38/20; A61K 39/395; A61P 35/00

(52) U.S. Cl. CPC A61K 31/426; C07K 16/2818; A61K 35/13; A61K 35/17; A61K 2039/5152; A61K 38/2026; A61K 39/395; A61P 35/00; A61K 2300/00; A61K 38/193

(57) ABSTRACT

The present invention provides methods of treating cancer.

### RELATED APPLICATIONS

This application claims priority to, and the benefit of U.S. Provisional Application No. 62/047,467 filed on Sep. 8, 2014, and U.S. Provisional Application No. 62/055,234 filed on Sep. 25, 2014, the contents of each of which are incorporated by reference in their entirety.

### GOVERNMENT INTEREST

This invention was made with government support under RO1CA143083 awarded by the National Cancer Institute. The government has certain rights in the invention.

### FIELD OF THE INVENTION

The present invention relates generally to treating cancer.

### BACKGROUND OF THE INVENTION

The cancer surveillance hypothesis was formalized in 1957 by Burnet and Thomas. It postulates a) the existence of tumor antigens “foreign” to the immune system due to somatic mutations or viral products, b) cancer surveillance by the immune system restricting the growth of the tumor and c) the idea of cancer immunotherapy. The corollary to these postulates (even though no research was conducted on the topic at the time) is that progressive tumors have been immunoedited and have evolved mechanisms for immune evasion.

The existence of tumor associated antigens, the ability of the immune system to mount an anti-tumor immune response and paradoxically even our knowledge of the mechanisms of immune evasion, all suggest that tumor immunotherapy is a feasible strategy. The promise of immunotherapy is tumor-restricted cytotoxicity and induction of immune memory; such that a cure for cancer can be envisioned instead of just prolonging patient life with the debilitating effects of conventional therapies.

A need exists to identify tumor immunotherapies.

### SUMMARY OF THE INVENTION

In various aspects the invention includes a method of increasing the efficacy of a cancer treatment regimen in a subject by administering to a subject receiving an active immunotherapy a PPAR gamma agonist.

In another aspect the invention includes a method of treating a cancer in a subject by administering to the subject a PPAR gamma agonist and an active immunotherapy.

In a further aspect the invention includes a method of reducing the number of T regulatory cells (Tregs) in a subject in need thereof by administering to the subject a PPAR gamma agonist. The subject has cancer. The subject is receiving an active immunotherapy treatment, an immune checkpoint inhibitor or both.

The active immunotherapy is a non-specific active immunotherapy or a specific active immunotherapy. The non-specific active immunotherapy is a cytokine. The cytokine is GM-CSF, MCSF or IL-4. The GM-CSF is administered via GM-CSF secreting cell or attached to a polymer scaffold. The specific active immunotherapy is adoptive T cell therapy or a tumor associated antigen vaccine. T-cell therapy is a chimeric antigen receptor T-cell (CART).

In some aspects the subject is further administered an immune check point inhibitor. The immune checkpoint inhibitor is an antibody specific for CTLA-4, PD-1, PD-L1, PD-L2 or killer immunoglobulin receptor (KIR). Non-limiting examples of immune checkpoint inhibitors include ipilimumab, tremelimumab pembrolizumab, nivolumab, pidilizumab, MPDL3280A, MEDI4736, BMS-936559, MSB0010718C, and AMP-224. The PPAR gamma agonist is a thiazolidinedione such as rosiglitazone (Rosi), pioglitazone, troglitazone, netoglitazone, or ciglitazone. The cancer is melanoma, non-small cell lung carcinoma (NSCLC), small cell lung cancer (SCLC) bladder cancer or prostate cancer.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTIONS OF THE DRAWINGS

FIG. 1: Isoforms and domains of full length PPAR-γ [1].

FIG. 2: Expression of PPAR-γ in B16 cells and various tissues. Lysates were made from the indicated tissue and analyzed for PPAR-γ expression. B-actin expression for normalization.

FIG. 3: Detection of overexpressed and endogenous PPAR-γ protein confirmed a requirement for GM-CSF to maintain PPAR-γ expression in alveolar macrophages. Alveolar macrophages from 2-wk old mice were collected by bronchoalveolar lavage (BAL) to reduce the confounding effects of proteinosis seen in older animals. The entire contents of the BAL from each mouse were lysed and loaded in a single lane. 3 WT and 3 GM-CSF−/− animals are shown above and B16 cells transduced with each of the two PPAR-γ isoforms were used as positive controls.

FIG. 4: PPAR-γ expression in resting peritoneal macrophages 5 hours after plating. Peritoneal cells were collected by a lavage and then plated for 5 hours. Non-adherent cells were washed off and the adherent cells were lysed in situ. Each lane represents one mouse.

FIG. 5: PPAR-γ expression in thioglycollate elicited peritoneal macrophages. Peritoneal cells were collected by a lavage and CD19 depleted. Remaining cells were lysed and lysates from individual mice were loaded in each lane.

FIG. 6: PPAR-γ expression in perigonadal adipose tissue. Adipose tissue was mechanically crushed in lysis buffer to obtain the lysate. Each lane represents an individual mouse.

FIG. 7: PPAR-γ expression in CD11b depleted splenocytes. Each lane represents an individual mouse.

FIG. 8: Detection of PPAR-γ by flow cytometry. A. Detection of overexpressed PPAR-γ in B16 cells. Detection of endogenous PPAR-γ in alveolar macrophages.

FIG. 9: Generation of myeloid specific KO of PPAR-γ. Peritoneal lavage was collected and plated for 2-4 hours. Non adherent cells were washed off and lysates were made from adherent cells. Expression of β-actin was used for normalization.

FIG. 10: Genetic depletion of PPAR-γ in myeloid cells reduces vaccination efficiency in B16 murine melanoma model. A. Schematic of prophylactic vaccine regimen. B. Survival curves of WT and PPAR-γ KO mice. Note that similar results were obtained when “fl only” or “cre only” mice were used as controls. 4 repeats were performed (total vaccinated con n=27, KO n=25). Some KO were protected but KO cohorts always displayed reduced protection against tumor challenge as compared to control C. Tumor incidence on day 60 after tumor challenge. D. Survival on day 60 after tumor challenge (not statistically significant). FIGS. 10E and 10F depict the effect of LysM (Lysin Motif) mediated conditional deletion of PPAR-g on GVAX efficacy. FIG. 10E is a graph that depicts GVAX treated KO mice show increased tumor incidence. FIG. 10F depicts reduced KO mice survival when compared to treated control (con) mice.

FIG. 11: CD expression remains unchanged in naïve PPAR-γ KO spleen. Spleens were mechanically digested and stained for CD11c, CD11c, CD19, CD and a dye to discriminate dead cells. Live cells were used to gate on the indicated populations.

FIG. 12: CD expression remains unchanged in vaccinated PPAR-γ KO spleens. Spleens were mechanically digested and stained for CD11c, CD11c, CD19, CD and a dye to discriminate dead cells. Live cells were used to gate on the indicated populations.

FIG. 13: Alveolar macrophages from PPAR-γ KO mice retain equivalent surface expression of CD1d. BAL was stained for flow cytometry and alveolar macrophages were identified by CD11c expression and co-labeled with CD1d. Our studies could not address a defect in CD1d expression in the APC recruited to the vaccine site, as these are technically challenging to harvest and then study by flow cytometry. Thus we used the live-B16 GM vaccine model where continuous release of GM-CSF and a palpable vaccine site allow easy harvest of recruited APC.

FIG. 14: A granulocytic, a monocytic and one DC population can be distinguished at the live-GM vaccine site in equal numbers in con and PPAR-γ KO mice. Over 25 control animals and approximately 12 PPAR-γ KO animals were examined. Gr-1 discrimination was conducted on 4 animals, in others CD14 was used to distinguish the monocytic fraction of the CD11b SP.

FIG. 15: No difference was detected in activation status of live-GM vaccine site granulocytes, monocytes and DC in PPAR-γ KO. MHCII (left), CD80 (middle) and CD86 (right) staining on DP cells (top two histograms), monocytes (middle two histograms) and granulocytes (bottom two histograms) from con (red) and KO (blue) vaccine sites.

FIG. 16: CD1d expression on CD11b SP and CD11b CD11c DP cells recruited to vaccine site was not affected in the PPAR-γ KO mice. Vaccine sites were processed on dl 1-d14. 4-7 animals were processed per group.

FIG. 17: PD-L1 expression on myeloid cells recruited to the vaccine site is not affected in the PPAR-γ KO. PD-L1 staining on DP cells (top two histograms), monocytes (middle two histograms) and granulocytes (bottom two histograms) from con (red) and KO (blue) vaccine sites.

FIG. 18: Subsets of APC recruited to the vaccine site. At least 6-8 mice were analyzed for con and KO each.

FIG. 19: Coculture with naïve or vaccinated CD4 and CD8 live vaccine site APC did not reveal a defect in the PPAR-γ KO. Myeloid cells were collected from B16-GM tumors using magnetic beads and cultured with splenic CD4 and CD8 cells from previously vaccinated or naive mice. A. CFSE dilution of FoxP3+ and FoxP3− CD4 and CD8. B. Cytokine production by CD4. C. Cytokine production by CD8. 50,000 APC were cultured with 500,000 T cells. 7-9 mice were tested per group across 3 experiments.

FIG. 20: NKT cells cultured with con or PPAR-γ KO vaccine site APC display similar cytokine profiles. 50000 APC from live-GM vaccine sites were cultured with 50000 24.8 NKT cell clone or Vb7 expressing primary NKT from somatic nuclear transfer mice for 48 hours. For aGC loading, APC were incubated with 500 ng/ml aGC for 2-4 hours and then washed repeatedly.

FIG. 21: GSEA shows difference in KO dLN consistent with loss of PPAR-γ in myeloid cells. dLN were collected 5 days after GVAX and analyzed by RNA-Seq. GSEA was performed to check for enrichment for all modules present in the Immgen database. A. Geneset known be induced by PPAR-γ in myeloid cells. B. Genesets known to be repressed by PPAR-γ.

FIG. 22: GSEA and flow cytometry show increased Treg and decreased CD8:FoxP3 ratio in PPAR-γ KO dLN. A Immgen modules enriched in Treg are shown in red with corresponding p-values for enrichment in KO dLN. B. Representative comparison of con and KO dLN and their CD8:Treg ratio by flow cytometry 6-8 days after vaccination. C. Quantification of LN CD8:Treg ratio. ˜25 mice each were evaluated for con and KO mice in 5 experiments.

FIG. 23: Analysis of tumor infiltrating leukocytes reveals lower T-cell infiltration in tumors in PPAR-γ KO mice. Con or KO females were challenged with live B16 cells (10̂5) and vaccinated with irradiated, GM-CSF secreting B16 cells (10̂6) at a different site on day one. Tumors were harvested on day 14, weighed, and processed to single cell suspensions, which were then stained with antibodies to CD45 and CD3. Tumor cells were excluded based on size/scatter profiles and lack of CD45 staining. 8-12 mice were studied per group. FIG. 23A depicts a timeline of therapeutic vaccination for tumor challenge and analysis. FIG. 23B is a series of graphs that depict tumor weight and characterization of the cellular population.

FIG. 24: The ratio of CD8+ T cells to FoxP3+ regulatory cells is decreased in tumors from vaccinated PPAR-γ KO animals. Con or KO females were challenged with live B16 cells (10̂5) and vaccinated with irradiated, GM-CSF secreting B16 cells (10̂6) at a different site on day one. Tumors were harvested on day 14, weighed, and processed to single cell suspensions, which were then stained with antibodies to CD45 and CD3. Tumor cells were excluded based on size/scatter profiles and lack of CD45 staining. 8-12 mice were studied per group. FIG. 24A depicts a timeline of therapeutic vaccination for tumor challenge and analysis. FIG. 24B is a series of graphs that depict characterization of the cellular population.

FIG. 25: KO dLN produce higher levels of Treg attracting chemokines. dLN were collected at the indicated time after GVAX. 5×10̂5 cells were plated and supernatants collected after 48 hours. Chemokine levels were measured by ELISA. Each data point represents a technical replicate. 3-4 mice were tested per group for each timepoint and sex. A paired comparison was performed on the 5 means (sex and time) for con and KO each to obtain the p-value.

FIG. 26: Con and KO CD8 from GVAX dLN produce equivalent levels of IFN-γ in response to Trp-2 peptide. 3-4 LN were pooled and 500,000 lymphocytes plated with 10 ug/ml of indicated peptide. Supernatants collected at 48 hours were assayed by ELISA. Data representative of 3 experiments.

FIG. 27: KO LN have increased expression of a Langerhans Cell specific gene module. dLN were collected 5 days after GVAX and analyzed by RNA-Seq. GSEA was performed to check for enrichment for all modules present in the Immgen database.

FIG. 28: LC express modest levels of lysozyme M. The Gene Skyline data viewer in Immgen was used to visualize Lysozyme M expression in key leukocyte populations.

FIG. 29: Staining strategy for Langerin expressing DC in the lymph node. Lymph nodes were mechanically digested to obtain single cell suspensions. Gated on live B220-MHCIIhi cells.

FIG. 30: Total CD207+ cells or the frequency of CD103 expression is unaffected in the PPAR-γ KO. At least 14 mice each were analyzed for con and KO LC across 4 experiments.

FIG. 31: Rosi does not impact the balance between CD8 and Treg in the vaccine draining lymph node after 6-8 days of treatment. Data representative of 3 experiments with 4-5 mice per group.

FIG. 32: 20 mg/kg/day Rosi delivered via drinking water improves the intratumoral CD8:Treg ratio in GVAX treated mice. Mice were challenged with 10A5 live tumor cells (left flank) and vaccinated with 10̂6 irradiated B16-GM cells (abdomen). Rosi or DMSO were added to their drinking water for 12 days. Tumors were harvested on day 14. Data pooled from 2 experiments. Each data point represents one mouse.

FIG. 33: Rosi mediated improvement in immune correlates requires PPAR-γ expression in myeloid cells. PPAR-g agonist Rosi improves intratumoral CD8:Treg ratio, the efficacy of GVAX+ anti-CTLA-4 combinatorial anti-tumor immunotherapy and promotes viral clearance in vaccinia infected mice. FIG. 33A depicts graphs from experiments in which mice were challenged with 10̂5 live tumor cells (left flank) and vaccinated with 1×10⁶ irradiated B16-GM cells (abdomen). Rosi or DMSO were added to their drinking water for 12 days. Tumors were harvested on day 14. Data pooled from 2 experiments. Each data point represents one mouse. FIG. 33B depicts the effect of Rosi on the survival of GVAX treated mice with B16 melanomas (top panel), the effect of Rosi on the incidence of B16 tumors in GVAX+anti-CTLA-4 treated tumors (middle panel), and the effect of Rosi on the survival of GVAX+anti-CTLA-4 mice with B16 melanomas.

FIG. 34: Rosi potentiates the efficacy of GVAX+CTLA-4 treatment. As described in methods, mice received challenge and vaccination (3×10̂6) on the same day. Rosi treatment was given for 12-14 days via drinking water (20 mg/kg/day). Anti-CTLA4 or isotype were injected i.p. on d0 (200 ug), d3 (100 ug) and d6 (100 ug).

FIG. 35: Treatment of human PBMC with GM-CSF and PPAR-γ modulators recapitulates Treg effects seen in murine studies. A. Two representative donor PBMC treated with Rosi. B. Treg numbers quantified for each donor. Each data point represents one donor. C. Effect of PPAR-γ inhibition on Treg numbers in human PBMC cultures treated with GM-CSF.

FIG. 36: CCL17 expression by GM-CSF treated human monocytes is reduced upon Rosi treatment. 1×10⁶ CD14+ human PBMC were cultured for 5 days with GM-CSF with 10 uM Rosi or vehicle control and CCL17 was measured by ELISA. CCL17 levels were normalized to the number of monocytes per well. Number of monocytes did not differ between con and Rosi treated wells.

FIG. 37: Analysis of adherent PBMC treated Rosi did not result in changes in number or activation status. Each data point represents a donor. Analysis was performed after 4-5 days of culture.

FIG. 38: Impact of PPAR-g deletion of DC related genes and function in MLR (mixed lymphocyte reactions). A. Expression of several genes associated the KEGG signaling “PPAR-g signaling” are reduced in the KO dLN. B. Expression of module 296 is reduced in KO dLN. C. DC associated genes are dysregulated in KO.

FIG. 39: KO LN DC retain a naïve migratory DC signature and support reduced survival of CD8 in MLR. FIG. 39A depicts increased expression of the gene signature of naïve migDC in KO LN. Balb/c splenocytes cultured with KO DC show reduced proliferation with a significant impact on total CD8 T cell numbers (FIGS. 39B and 39C).

FIG. 40: T cell defects in GVAX draining LN of Lys-M-Cre; PPAR-g fl mice. FIG. 40A depicts that the Expression of Treg associated genesets is increased in KO dLN. FIG. 40B depicts flow cytometry plots of dLN. FIG. 40C depicts the quantification of LN cellularity, CD8 frequency and CD8:Treg ratio. Each data point represents one mouse. FIG. 40D is a graph that depicts that the expression of Treg recruiting chemokines is increased in KO dLN. FIGS. 40E and 40F are a series of graphs that depict CD8 number (assessed by flow cytometry) and CCL22 expression (assessed by ELISA) obtained from LN from vaccinia scarred mice that were cultured for 4 days.

FIG. 41: Treg from GVAX treated mice express high levels of coinhibitory receptors TIGIT and CTLA4. FIG. 41A is a flow cytometry plot of TIGIT expression. FIG. 41B is a flow cytometry plot of CTLA-4 expression in naïve LN (FIGS. 41A and 41B) and LN harvested 6-8 days after GVAX (FIGS. 41C and 41D).

FIG. 42: PPAR-g agonist Rosi reduces ulceration of Lewis Lung Carcinoma in combination with GVAX+anti-CTLA4 and improves survival. FIG. 42A depicts the effect of Rosi on the ulceration of subcutaneous Lewis Lung Carcinomas in GVAX+anti-CTLA-4 mice. FIG. 42B depicts the effect of Rosi on the survival of GVAX+anti-CTLA-4 mice with ectopic subcutaneous Lewis Lung Carcinomas.

FIG. 43: Role of PPAR-g in restraining Treg recruitment and expansion is conserved in GM-CSF treated human monocytes. FIGS. 43A and 43B depict the effect of PPAR-g agonist Rosi and PPARg inh on CCl17 (FIG. 43A) and CCl22 (FIG. 43B) in human monocytes cultured with a GM-CSF expressing cell line.

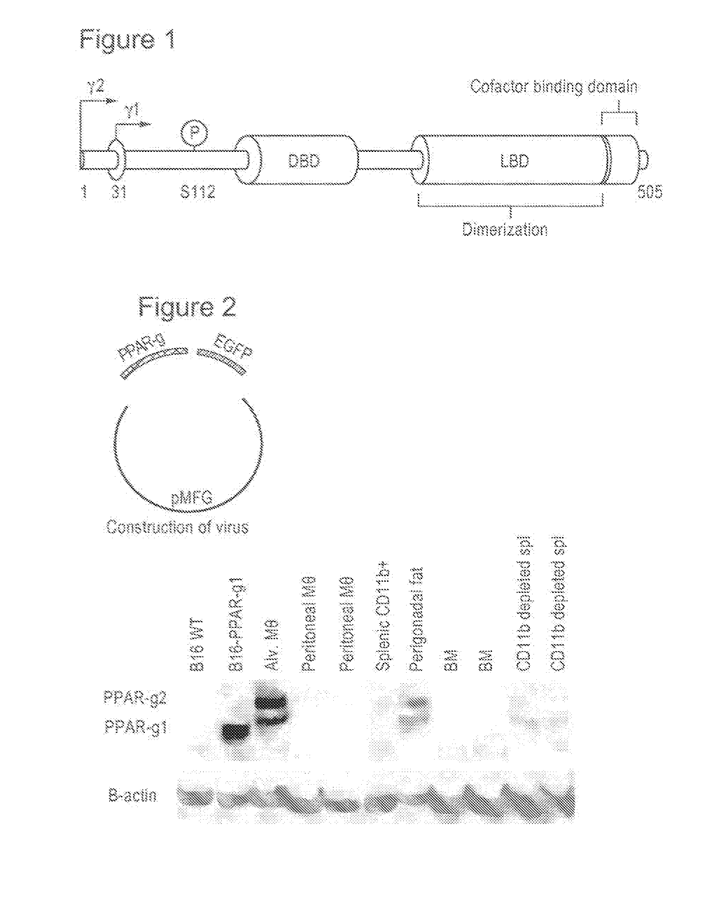

### DETAILED DESCRIPTION OF THE INVENTION

The present invention is based in part upon the suprising discovery that PPAR-γ is required for protective immunity stimulated by cancer vaccines. Administration of PPAR-γ agonists in combination with immunotherapy resulted in greater therapeutic effects. Additionally, administration reduces the generation of T regulatory cells (Tregs)

Granulocyte macrophage colony stimulating factor (GM-CSF) mediates context dependent anti- or pro-inflammatory functions through cells of the myeloid lineage. GM-CSF signaling induces the expression of the transcription factor peroxisome proliferator-activated receptor gamma (PPAR-γ). We examined the role PPAR-γ in myeloid cells in the anti-tumor response to GVAX, a GM-CSF (granulocyte-macrophage colony-stimulating factor) based cancer immunotherapy using the B16 model of murine melanoma. GVAX is a GM-CSF tumor cell vaccine. GVAX makes use of autologous or allogeneic tumor cells as immunogens; in this approach, the tumor cells are genetically modified to express GM-CSF.

It was discovered that selective loss of PPAR-γ in the myeloid lineage using LysM-Cre reduces the efficacy of GVAX which could not be explained by known mechanisms. LysM (Lysin Motif) is a protein motif that noncovalently binds to peptidoglycan and chitin via interactions with N-acetylglucosamine moieties. RNASeq of GVAX draining lymph node identified an increase in regulatory T-cells markers such as FoxP3 and coinhibitory receptors CTLA-4 and TIGIT in LysM-Cre; PPAR-γ fl mice (PPAR-γ KO). Flow cytometry confirmed that Treg frequency was indeed increased in PPAR-γ KO lymph node with a strong reduction seen in the ratio of CD8 T-cells to regulatory T cell (CD8:Treg). Treg recruiting chemokines CCL17 and CCL22 were upregulated in the draining lymph node. Importantly, tumors in PPAR-γ KO mice had a reduced CD8:Treg ratio explaining the loss in GVAX efficacy.

Pharmacological activation or inactivation of PPAR-γ in GM-CSF treated human PBMC showed conservation of the role of PPAR-γ in regulating T-cell numbers in humans. PPAR-γ agonism in mice, using the FDA-approved small molecule ligand rosiglitazone (Rosi), improved CD8:Treg ratios in the vaccine draining lymph node and tumors. The gain-of-function data suggested the Rosi could be used as an adjunct to immunotherapy. All intratumoral Treg expressed high levels of CTLA-4 and TIGIT. Thus, we tested the impact of Rosi on the response to GVAX and anti-CTLA-4 combination therapy. We found that Rosi improved the tumor incidence and overall survival of tumor bearing mice treated with GVAX and anti-CTLA4.

Our data have identified a novel role of PPAR-γ in myeloid cells in regulating Treg numbers. This pathway is conserved in humans as seen in ex-vivo studies of PBMC. Further, we provide preclinical evidence that Rosi can be used to improve immunotherapeutic responses by increasing the ratio between intratumoral effector and regulatory cells.

Accordingly, the invention provides methods of increasing the efficacy a cancer treatment regimen in a subject by administering to a subject receiving an active immunotherapy a PPAR gamma agonist.

Additionally, the invention includes a method of treating a cancer in a subject by administering to the subject a PPAR gamma agonist and an active immunotherapy.

In a further aspect the invention includes a method of reducing the number of T regulatory cells (Tregs) in a subject in need thereof by administering to the subject a PPAR gamma agonist.

The data presented in the Example section show an unexpected requirement for PPAR-g expression in LysM expressing cells in maintaining GVAX efficacy. CCL22 upregulation in the KO and impact on CD8 numbers is conserved in vaccinia draining LN. Further, in human monocytes also CCL17 and CCL22 are downregulated by PPAR-g activation and Treg numbers are reduced in co-culture. Thus, the explored phenotypes are conserved in murine models of cellular vaccination (GVAX), viral vaccination (vaccinia) and in human monocytes.

Using a combination of high throughput analysis of gene signatures and functional assays we identify defects in the LN dendritic cells. The persistence of a naïve migratory DC gene signature suggests that some of the functional defects are found in migDC subsets. While monocytes are the most widely studied subset in LysM-Cre mice, it is known to affect some classical DC subtypes and neutrophils. Further, under conditions of inflammation, monocytes can repopulate antigen carrying DC migrating from the skin. Thus it is possible that our findings reflect a cell intrinsic defect in dendritic cells. However, another hypothesis is that LN macrophages are defective causing the immigrant DC to be less activated. We cannot preclude defects in other cell types in addition to LN DC. In fact we find several macrophage genes such as CD163, CD169, neutrophil genes such as elastase and neutrophilic granule protein and genes encoding mast cell proteases impacted in the KO LN. In future studies, we hope to isolate and explore the complex defects in the KO dLN.

The KO mice have defects in the T-cell response to GVAX. Importantly, the CD8:Treg ratio at the tumor site is reduced. Sato et al published the first clinical data to show that the balance between cytolytic and regulatory T-cells allowed clear stratification and correlation with patient response to therapy compared to absolute numbers of cytolytic cells. Since then, multiple studies, including a Phase I trial of GVAX in combination with anti-CTLA4, have found the CD8:Treg ratio to be prognostic for many cancers. While we provide the first evidence of the cellular changes underlying the decreased Treg on Rosiglitazone treatment, it is interesting to note that Treg numbers were previously shown to be reduced by Rosi in combination with Gemcitabine, a compound known to target myeloid derived suppressor cells.

The reduced CD8:Treg ratio correlates with the reduced T cell survival in culture and the increased expression of Treg recruiting chemokines. We propose a model where the antigen specific T-cells have defective survival, yet the Treg numbers increase due to increased recruitment. CCL17 and CCL22 have been frequently implicated in recruiting Treg. However, their relative effects on recruiting various T cell subsets are context dependent. CCL17 for instance, is known to reduce rather than recruit Treg in atherosclerosis. It has also been linked to an improved Th2 response. CCL17 has independently been detected as a GM-CSF and PPAR-γ dependent gene in gene expression analyses. Most ex-vivo studies of CCL17 function are conducted on GM-CSF derived dendritic cells. Interestingly, CCL17 was found to be an indicator of better prognosis in a tumor vaccine study where the patients were administered GM-CSF in addition to a peptide vaccine. However this effect was only seen in patients treated with cyclophosphamide, a Treg modulation agent. Given CCL17's apparently conflicting effects on helper T-cells as well as regulatory T cells, one hypothesis to reconcile the above data would be that CCL17 has immunostimulatory functions in addition to induction of Treg; and the former dominate once Treg are suppressed. Previously described is a GM-CSF dependent upregulation of CCL22 and induction of Treg from dendritic cells treated with apoptotic thymocytes. Thus, it is not surprising that CCL22 mediated Treg induction should play a role in the vaccine response induced by a GM-CSF dependent cellular vaccine. To our knowledge, CCL22 has not previously been linked to PPAR-γ. CCL17 secretion has only been seen in myeloid cells. The producers of CCL22 are generally also of myeloid origin. However in rare studies, CCL22 has been shown to be expressed by CD8 cells and NK cells.

With GVAX alone, Rosi improved CD8:Treg ratio but had no impact on tumor size and survival. However, it showed a clear benefit in combination with GVAX and anti-CTLA4. Several studies have shown that while treatment with a single antibody against coinhibitory molecules is insufficient, double blockade or Treg depletion can lead to successful regression in murine tumor models. Combinatorial immunotherapy is the emerging standard in clinical practice also. Sequentially delivery of GVAX and anti-CTLA4 has been tested in patients and our data suggests that a triple combination of Rosi, GVAX and anti-CTLA-4 could achieve significant benefits. These findings are exciting because, anti-CTLA4 (Ipilimumab) is a recently approved immunotherapy.

Without being bound to any specific mechanism, theory or hypothesis, several hypotheses may be formed regarding why Rosi impacts tumor growth only in the presence of anti-CTLA-4. Tumors have many redundant mechanisms for immune evasion. Thus, it is possible that despite a favorable CD8:Treg ratio, the CD8 are dysfunctional till CTLA4 is blocked. CTLA4 blockade could be playing a CD8 intrinsic role or through its expression on the persisting Treg or even tumor resident myeloid cells. Further, Rosi provides a first-in-class therapy to target intratumoral Treg in patients. In mice, several strategies exist to target Treg including anti-CD25 but none to target Treg in the clinic. CD25 blockade is impractical for clinical use as it could affect the CD25 levels on effector T-cells.

Testing KO mice and Rosi treatment with other vaccination approaches, both cellular and non-cellular will allow further elucidation of the immunostimulatory roles of PPAR-γ and its therapeutic value. One synergy that could be explored is with blockade of the PD-1/PD-L1 pathway in combination with Rosi treatment. PD-1 is expressed in TILS in GVAX treated mice, yet PD-1 did not emerge as a differentially expressed gene in the RNASeq of KO dLN. Combination with PD-1 blockade will inform our understanding of the mechanisms of synergy between Rosi and checkpoint blockade. A coinhibitory receptor that is elevated in the KO dLN is the newly identified TIGIT. Treg from GVAX dLN or tumor sites (from con or KO animals) are TIGIT positive. Given our data on Treg in GVAX and role of PPAR-γ, the triple combination of GVAX, TIGIT blockade and Rosi appears to be a promising avenue to explore.

The defect in vaccine response and the T-cell and DC phenotypes are partial but significant. It is important to note that PPAR-γ has known immunosuppressive effects. In keeping with the previously published suppression of IFN-g response by PPAR-g, the geneset for IFN-g induced genes was upregulated in the KO. Hence, the modest defects that we see are the sum of immunosuppressive and pro-tumor immunity effects of PPAR-γ. This implies that systemic Rosi treatment could have cell dependent pro or anti-inflammatory effects. Future studies are planned to provide Rosi locally or target it to known cell types.

Therapeutic Methods

The efficacy of a cancer treatment is increased administering to a subject a PPAR-γ agonist. The subject is receiving an active immunotherapy. The PPAR-γ agonist may be administered concurrently, prior to or after the subject receives an active immunotherapy treatment. In some aspects the subject is further administered an immune check point inhibitor. Also included in the invention are methods of reducing the number of T regulatory cells (Tregs) in a subject in need thereof by administering to the subject a PPAR-γ agonist. Subjects in need thereof includes subjects who have cancer, are receiving an active immunotherapy treatment and/or an immune checkpoint inhibitor.

The methods described herein are useful to alleviate the symptoms of a variety of cancers.

Treatment is efficacious if the treatment leads to clinical benefit such as, a decrease in size, prevalence, or metastatic potential of the tumor in the subject. When treatment is applied prophylactically, “efficacious” means that the treatment retards or prevents tumors from forming or prevents or alleviates a symptom of clinical symptom of the tumor. Efficaciousness is determined in association with any known method for diagnosing or treating the particular tumor type.

PPAR Gamma Agonists

Peroxisome proliferator-activated receptor gamma (PPAR-γ or PPARG), also known as the glitazone receptor, or NR1C3 (nuclear receptor subfamily 1, group C, member 3) is a type II nuclear receptor that in humans is encoded by the PPARG gene. Two isoforms of PPARG are detected in the human and in the mouse: PPAR-γ1 (found in nearly all tissues except muscle) and PPAR-γ2 (mostly found in adipose tissue and the intestine).

PPARG regulates fatty acid storage and glucose metabolism. The genes activated by PPARG stimulate lipid uptake and adipogenesis by fat cells. PPARG knockout mice fail to generate adipose tissue when fed a high-fat diet.

A PPAR-γ agonist is a compound that binds to a receptor and activates the receptor to produce a biological response.

The PPAR-γ agonist can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

For example, the PPAR-γ agonist is a thiazolidinedione. Preferably, the thiazolidinedione is rosiglitazone (Rosi), pioglitazone, troglitazone, netoglitazone, ciglitazone, netoglitazone, or rivoglitazone. In another aspect, the PPAR-γ agonist is saroglitazar, magnolol, honokiol, falcarindiol, resveratrol, amorfrutin 1, quercetin, or linolenic acid.

The PPAR-γ agonist is an antibody or fragment thereof that activates PPAR-γ. Methods for designing and producing agonist antibodies are well-known in the art.

Active Immunotherapy

Active immunotherapy attempts to stimulate the immune system by presenting antigens in a way that triggers an immune response.

For the immune system to garner a response against a tumor, the tumor must have an antigen that distinguishes it from the surrounding normal tissue.

There are two types of active immunotherapy; non-specific immunotherapy and specific immunotherapy

Non-Specific Active Immunotherapy generates a general immune system response using cytokines and other cell signaling. Cytokines include for example, GM-CSF and MCSF. The cytokines are delivered via a cell engineered to secrete the cytokine or the cytokine is attached to a polymer scaffold.

Specific Active Immunotherapy includes the generation of cell-mediated and antibody immune responses focused on specific antigens expressed by the cancer cells. Specific active immunotherapy includes for example antigen-specific vaccines, or adoptive transfer of anti-tumor T cells. Numerous platforms have been developed and evaluated clinically to induce immune responses against tumor-associated antigens. Antigen specific vaccination includes whole cell-based vaccines as well as peptides and whole protein-based approaches. As alternative approach antigen-specific vaccines includes raising the frequency of tumor-specific T cell populations by adoptive T cell transfer. Adoptive transfer of anti-tumor T cells bypasses the need for the endogenous host immune system to respond to an exogenous vaccine, and can involve delivery of enormous numbers of cells, offering a quantitative advantage. The approach also allows for direct manipulation of the T cell population being administered, and also conditioning of the host to support optimal T cell persistence and functional maintenance. Adoptive T-cell transfer includes the use of chimeric antigen receptor T-cells (CARTS)

Immune Checkpoint Inhibitors

Immune checkpoints refer to a plethora of inhibitory pathways hardwired into the immune system that are crucial for maintaining self-tolerance and modulating the duration and amplitude of physiological immune responses in peripheral tissues in order to minimize collateral tissue damage. It is now clear that tumors co-opt certain immune-checkpoint pathways as a major mechanism of immune resistance, particularly against T cells that are specific for tumor antigens. Because many of the immune checkpoints are initiated by ligand-receptor interactions, they can be readily blocked by antibodies or modulated by recombinant forms of ligands or receptors.

Immune checkpoints include CTIA-4, Pd-1, PD-L1, PD-L2, killer immunoglobulin receptor (KIR), LAG3, B7-H3, B7-H4, TIM3, A2aR, CD40L, CD27, OX40, 4-IBB, TCR, BTLA, ICOS, CD28, CD80, CD86, ICOS-L, B7-H4, HVEM, 4-1BBL, OX40L, CD70, CD40, and GAL9.

Non-limiting examples of immune checkpoint inhibitors include ipilimumab, tremelimumab pembrolizumab, nivolumab, pidilizumab, MPDL3280A, MEDI4736, BMS-936559, MSB0010718C, and AMP-224.

Therapeutic Administration

The invention includes administering to a subject, a composition containing an active immunotherapy compound, a PPAR-γ agonist, an immune checkpoint inhibitor or any combination thereof.

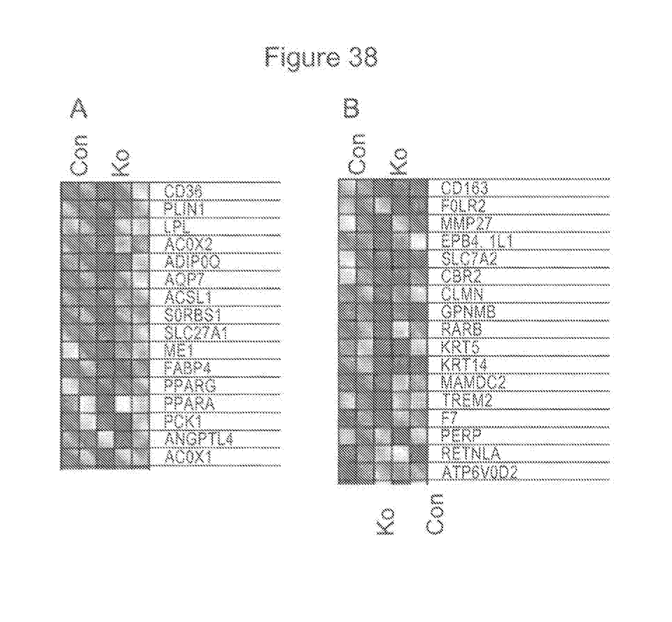

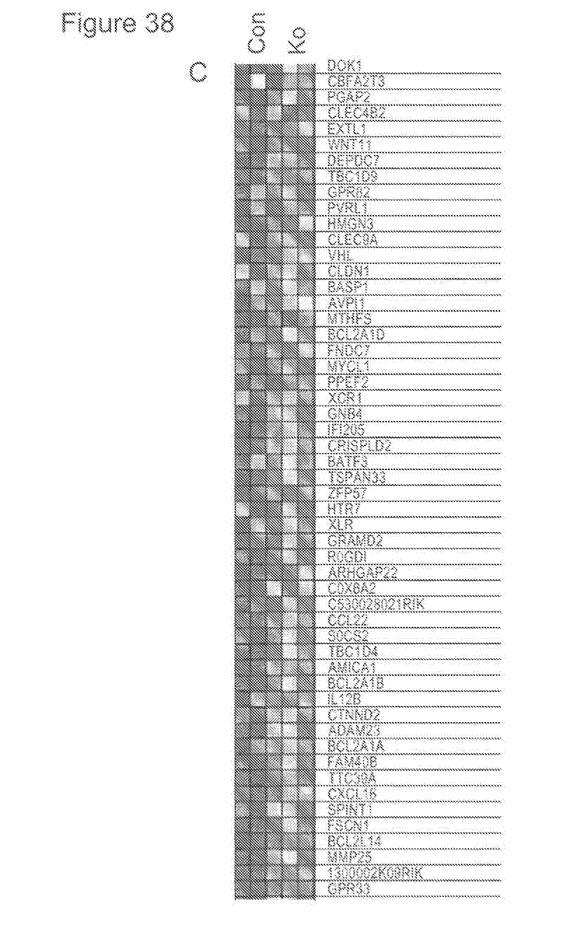

Alternatively, the invention includes administering to a subject an active immunotherapy compound, or an immune checkpoint inhibitor, or a compound that increases the expression of one or more genes that are downregulated in the PPAR-γ KO studies (see FIG. 38 for full list) such that the expression of the one or more downregulated genes becomes increased, or administering to a subject a compound that decreases the expression of one or more genes that are upregulated in the PPAR-γ KO studies (see FIG. 38 for full list) such that the expression of the one or more genes that are upregulated becomes decreased, or any combination thereof.

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other anti-proliferative agents or therapeutic agents for treating, preventing or alleviating a symptom of a cancer. A therapeutic regimen is carried out by identifying a mammal, e.g., a human patient suffering from a cancer using standard methods.

Doses may be administered once, or more than once. In some embodiments, it is preferred that the therapeutic compound is administered once a week, twice a week, three times a week, four times a week, five times a week, six times a week, or seven times a week for a predetermined duration of time. The predetermined duration of time may be 1 week, 2 weeks, 3 weeks, 4 weeks, 5 weeks, 6 weeks, 7 weeks, 2 months, 3 months, 4 months, 5 months, 6 months, 7 months, 8 months, 9 months, 10 months, 11 months, or up to 1 year.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The inhibitors are optionally formulated as a component of a cocktail of therapeutic drugs to treat cancers. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, the therapeutic compounds are formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, conventional filler, and a tableting agent. Other formulations include an ointment, suppository, paste, spray, patch, cream, gel, resorbable sponge, or foam. Such formulations are produced using methods well known in the art.

Therapeutic compounds are effective upon direct contact of the compound with the affected tissue. Accordingly, the compound is administered topically. Alternatively, the therapeutic compounds are administered systemically. For example, the compounds are administered by inhalation. The compounds are delivered in the form of an aerosol spray from pressured container or dispenser which contains a suitable propellant, e.g., a gas such as carbon dioxide, or a nebulizer.

Additionally, compounds are administered by implanting (either directly into an organ or subcutaneously) a solid or resorbable matrix which slowly releases the compound into adjacent and surrounding tissues of the subject.

In some embodiments, it is preferred that the therapeutic compounds described herein are administered in combination with another therapeutic agent, such as a chemotherapeutic agent, radiation therapy, or an anti-mitotic agent. In some aspects, the anti-mitotic agent is administered prior to administration of the present therapeutic compound, in order to induce additional chromosomal instability to increase the efficacy of the present invention to targeting cancer cells. Examples of anti-mitotic agents include taxanes (i.e., paclitaxel, docetaxel), and vinca alkaloids (i.e., vinblastine, vincristine, vindesine, vinorelbine).

### Definitions

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

Thus, treating may include suppressing, inhibiting, preventing, treating, or a combination thereof. Treating refers inter alia to increasing time to sustained progression, expediting remission, inducing remission, augmenting remission, speeding recovery, increasing efficacy of or decreasing resistance to alternative therapeutics, or a combination thereof “Suppressing” or “inhibiting”, refers inter alia to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. The symptoms are primary, while in another embodiment, symptoms are secondary. “Primary” refers to a symptom that is a direct result of the proliferative disorder, while, secondary refers to a symptom that is derived from or consequent to a primary cause. Symptoms may be any manifestation of a disease or pathological condition.

The “treatment of cancer or tumor cells”, refers to an amount of peptide or nucleic acid, described throughout the specification, capable of invoking one or more of the following effects: (1) inhibition of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

As used herein, “an ameliorated symptom” or “treated symptom” refers to a symptom which approaches a normalized value, e.g., is less than 50% different from a normalized value, preferably is less than about 25% different from a normalized value, more preferably, is less than 10% different from a normalized value, and still more preferably, is not significantly different from a normalized value as determined using routine statistical tests.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” or “therapeutic amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer to shrink rr or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head and neck, kidney, lung, non-small cell lung, melanoma, mesothelioma, ovary, sarcoma, stomach, uterus and Medulloblastoma. Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

A “proliferative disorder” is a disease or condition caused by cells which grow more quickly than normal cells, i.e., tumor cells. Proliferative disorders include benign tumors and malignant tumors. When classified by structure of the tumor, proliferative disorders include solid tumors and hematopoietic tumors.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

By the term “modulate,” it is meant that any of the mentioned activities, are, e.g., increased, enhanced, increased, augmented, agonized (acts as an agonist), promoted, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values.

As used herein, the term “administering to a cell” (e.g., an expression vector, nucleic acid, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

As used herein, “molecule” is used generically to encompass any vector, antibody, protein, drug and the like which are used in therapy and can be detected in a patient by the methods of the invention. For example, multiple different types of nucleic acid delivery vectors encoding different types of genes which may act together to promote a therapeutic effect, or to increase the efficacy or selectivity of gene transfer and/or gene expression in a cell. The nucleic acid delivery vector may be provided as naked nucleic acids or in a delivery vehicle associated with one or more molecules for facilitating entry of a nucleic acid into a cell. Suitable delivery vehicles include, but are not limited to: liposomal formulations, polypeptides; polysaccharides; lipopolysaccharides, viral formulations (e.g., including viruses, viral particles, artificial viral envelopes and the like), cell delivery vehicles, and the like.

### EXAMPLES

### Example 1: The Role of GM-CSF in Maintaining PPAR-Γ Expression in Myeloid and Non-Myeloid Cells

Methods

Virus Generation

cDNA encoding for each isoform of PPAR-γ was inserted into the retroviral vector pMFG using standard recombinant DNA technology. pMFG-PPAR-γ plasmid was transfected into a packaging cell line, 293GPG, which expresses the protein components necessary for viral assembly using Lipofectamine. Supernatants containing the secreted virus were collected starting on day 2 for several days. Virus particles were precipitated by high-speed ultracentrifugation, resuspended in OptiMem and stored in −80° C. till needed.

B16 Culture and Infection

B16 were cultured in DMEM containing 10% FCS and antibiotics. For infection, 1×10̂5-2×10̂5 B16 were plated and incubated with polybrene and concentrated virus. After 24 hours, cultures were washed and allowed to become confluent.

Lysate Preparation and Western Blot for Detection of PPAR-γ Protein

Depletion of CD11b cells was performed using magnetic beads (Miltenyi Biotec). Cells were lysed in the following: RIPA buffer containing 10% protease inhibitors (Sigma-Aldrich; Cat. No. P8340) and ImM Na3OV4 and PMSF Immediately after lysis, samples were sonicated briefly and then spun down at 15000 g for 15 min at 4° C. Supernatants were collected and heated to 70° C. with loading buffer containing lithium dodecyl sulfate and 100 mM DTT.

For Western Blotting, a rabbit anti-PPAR-γ antibody (Cell Signaling, 81B8) was used followed by an alkaline phosphatase conjugated secondary. A chemiluminescent substrate was used to develop the blot (Vector Labs, SK-6605). Densitometry was conducted using ImageJ software.

Detection of PPAR-γ by Western Blot

We required a robust assay to detect PPAR-γ to confirm its relationship with GM-CSF and myeloid specific loss in GM-CSF−/− mice. We generated B16 derived cell lines overexpressing each isoform and screened commercially available antibodies to select one with robust and specific detection of PPAR-γ (FIG. 2). We screened alveolar and peritoneal macrophages; and CD11b+ splenic cells. CD11b is expressed on monocytes and neutrophils. Thus this population includes splenic monocytes, macrophages, monocyte derived dendritic cells and neutrophils. We also tested perigonadal fat pads (as an endogenous positive control), total bone marrow and the bulk spleen left after CD11b fractionation. We were unable to detect PPAR-γ in splenic myeloid cells or freshly recovered peritoneal macrophages. Most importantly, endogenous PPAR-γ was detectable in alveolar macrophages using this antibody (FIG. 2). As reviewed in the introduction, GM-CSF mediates important aspects of alveolar macrophage biology and thus they represent an important cell type to study GM-CSF induced genes.

Results

PPAR-γ is a Target of GM-CSF in Alveolar Macrophages

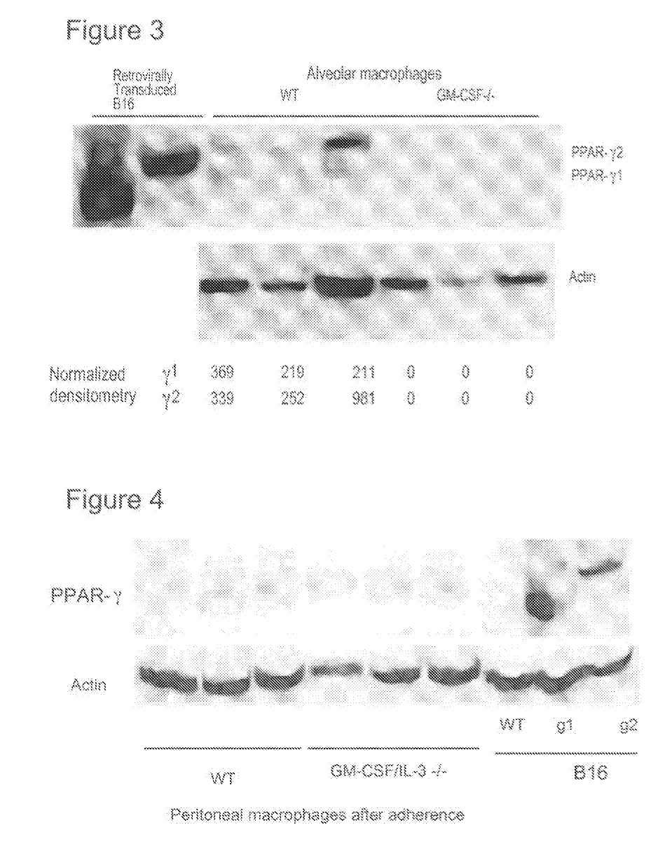

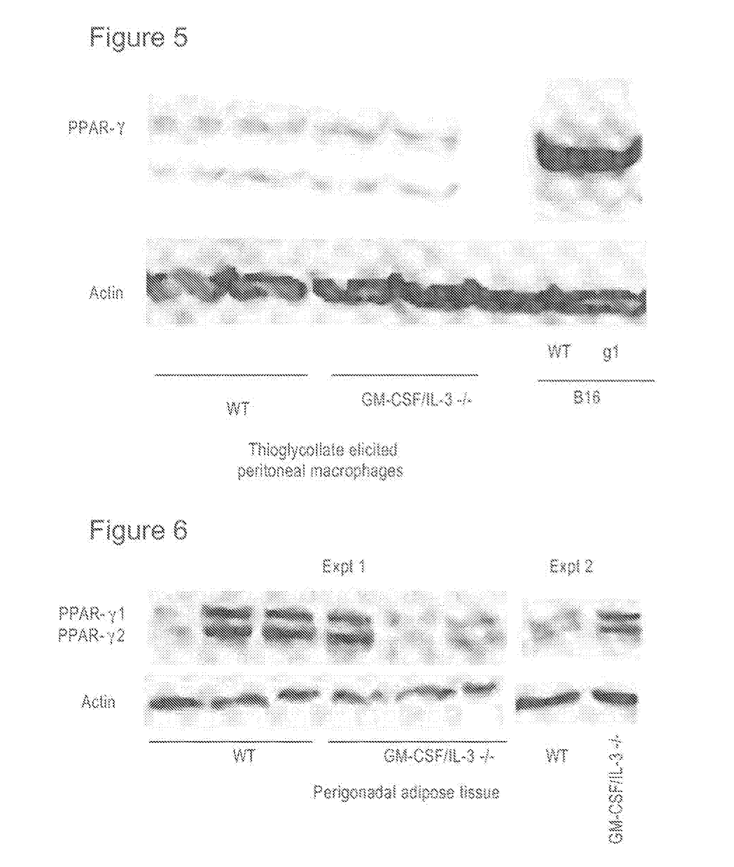

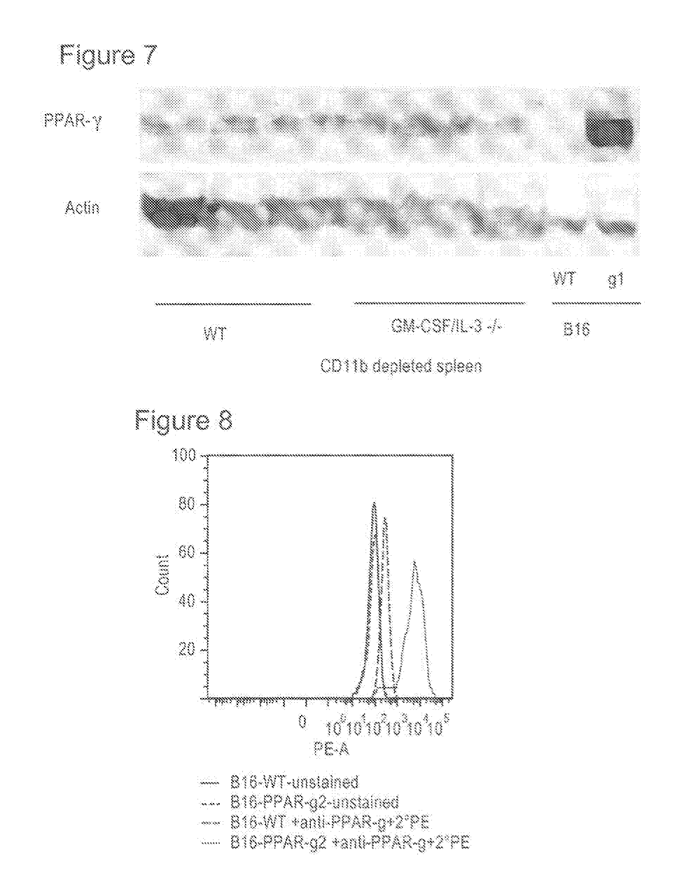

PPAR-γ expression in alveolar macrophages was previously shown to be GM-CSF dependent. We were able to confirm these findings. Alveolar macrophages from GM-CSF KO mice were completely deficient in PPAR-γ (FIG. 3). Interestingly, we found that freshly isolated peritoneal macrophages did not express detectable amount of PPAR-γ protein. Adherence to a tissue culture dish upregulated expression which was not GM-CSF dependent (FIG. 4). We also tested if thioglycollate elicitation would lead to PPAR-γ expression and its dependence on GM-CSF. Significant but GM-CSF independent PPAR-γ can be detected in peritoneal macrophages upon thioglycollate elicitation (FIG. 5). Further, PPAR-γ expression in perigonadal fat pad and CD11b depleted spleen was also GM-CSF independent (FIGS. 6 and 7). Two experiments are shown for perigonadal fat as expression was quite variable from mouse to mouse. These findings suggested that PPAR-γ is a GM-CSF target in certain macrophages and that differentiation or anatomical location conferred differential dependence on GM-CSF for PPAR-γ expression. In fat and lymphocytes (CD11b depleted spleen) which do not express GM-CSF receptor, PPAR-γ expression is expected to be independent of GM-CSF.

We also tested if PPAR-γ expression could be detected by flow cytometry. We were able to detect ectopic expression in B16 (FIG. 8) but were unable to detect endogenous expression in alveolar macrophages.

### Discussion

The above studies are a systematic analysis of PPAR-γ expression in various myeloid as well as selected non-myeloid tissues. Given the defects in apoptotic cell phagocytosis of various myeloid populations deficient in PPAR-γ, it is surprising that we were only able to detect PPAR-γ expression in alveolar macrophages. We find that homeostatic PPAR-γ expression in alveolar macrophages is entirely dependent on GM-CSF presence. GM-CSF has important roles to play in mucosal surfaces, thus it is possible that myeloid populations in the gut mucosa also show GM-CSF dependent PPAR-γ expression. Our data are in accordance with a recently published analysis of PPAR-γ mRNA expression in various myeloid subtypes. Gautier et al also found that peritoneal macrophages only expressed PPAR-γ under inflammatory conditions. To our knowledge, this is the first evidence of adherence leading to PPAR-γ expression in macrophages. Gautier et al also show that monocytes recruited to the site of inflammation express PPAR-γ. Thus, in the following chapter we tested if the immune response to GVAX was affected in LysM-Cre; PPAR-γ fl mice.

### Example 2: Effect of Genetic Loss-of-Function in the Monocyte Lineage on GVAX: Studies on Candidate Mechanisms

Methods

Generation of LysM-Cre; PPAR-γ fl

Commercially available LysM-Cre (Jackson Laboratory, 4781) and PPAR-γ fl (Jackson Laboratory, 4584) mice were crossed together. F1×F1 crosses lead to Cre and fl homozygous animals which were viable and fertile.

Restimulation of Splenocytes

Spleens were crushed, subjected to red blood cell lysis and passed through a 70 um strainer to obtain single cell suspensions. 2×10̂6 cells were plated in 2 ml of media with 50,000 irradiated B16 cells. Cytokine levels were measured after 4 days.

Tumor Processing

B16-GM tumors were harvested and weighed. Tumors were chopped into 1-3 mm pieces and incubated in media containing 200 units Collagenase IV and 10 ug/ml DNAse for 45-75 minutes at 37° C. After incubation, the tissue was pipetted repeatedly and strained with a 70 um strainer. A gradient for centrifugation was generated using Optiprep (Sigma-Aldrich). 25 ml of a solution containing 0.85% NaCl and 10 mM Tricine in distilled water was mixed with another 5 ml of distilled water and 8.71 ml of Optiprep. This gradient was layered under media containing the tumor single cell suspension and spun at 400 g for 25 minutes at RT with slow deceleration. The interface was collected and analyzed for flow cytometry or used for coculture.

Coculture Experiments

Naïve and vaccinated spleens were processed to single cell suspensions. CD8 were selected by using anti-CD8 labeled magnetic beads. Following that CD4 were recovered by negative selection, again using magnetic beads. 50,000 APC were incubated with 500,000 CD4 or CD8.

For NKT cell coculture, 50,000 APC were incubated with 50,000 24.8 or primary NKT from Vb7 somatic nuclear transfer mice. All CD4 in these mice are NKT cells. There are also CD4− NKT cells. To purify the primary NKT, a negative selection was performed for CD4 using magnetic beads. For aGC loading, APC were incubated with 500 ng/ml aGC for 2-4 hours and then washed repeatedly.

Results

LysM-Cre; PPAR-γ Fl Mice Show Significant Loss of PPAR-γ and Recapitulate the Lung Pathology of GM-CSF KO Mice

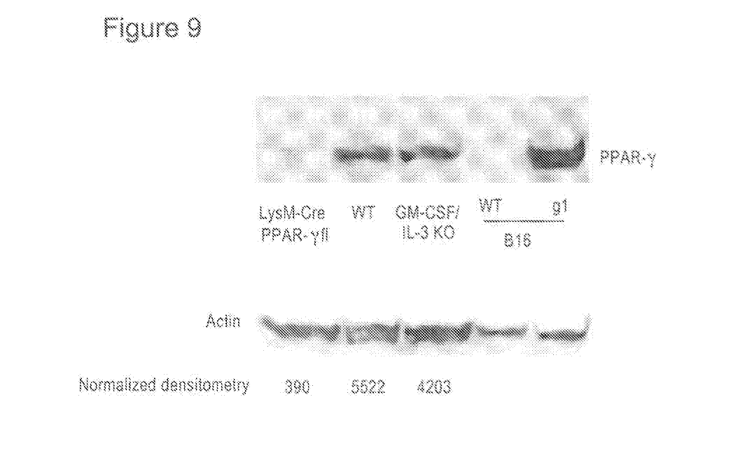

We crossed LysM-Cre mice to mice with loxP sites flanking the pparg locus. As shown in FIG. 9, peritoneal macrophages from PPAR-γ KO mice had a greater than 90% reduction in PPAR-γ protein expression. PPAR-γ KO mice showed some evidence of protein accumulation and inflammation in the BAL, and histologic analysis revealed mild pathologic changes consistent with pulmonary alveolar proteinosis (data not shown). It has been previously shown that proteinosis in PPAR-γ KO mice is not as severe as in GM-CSF deficient mice implying that PPAR-γ is only one of the downstream effectors involved in GM-CSF regulated surfactant homeostasis

PPAR-γ KO Mice Show Reduced Protection Against Tumor Challenge after Prophylactic GVAX

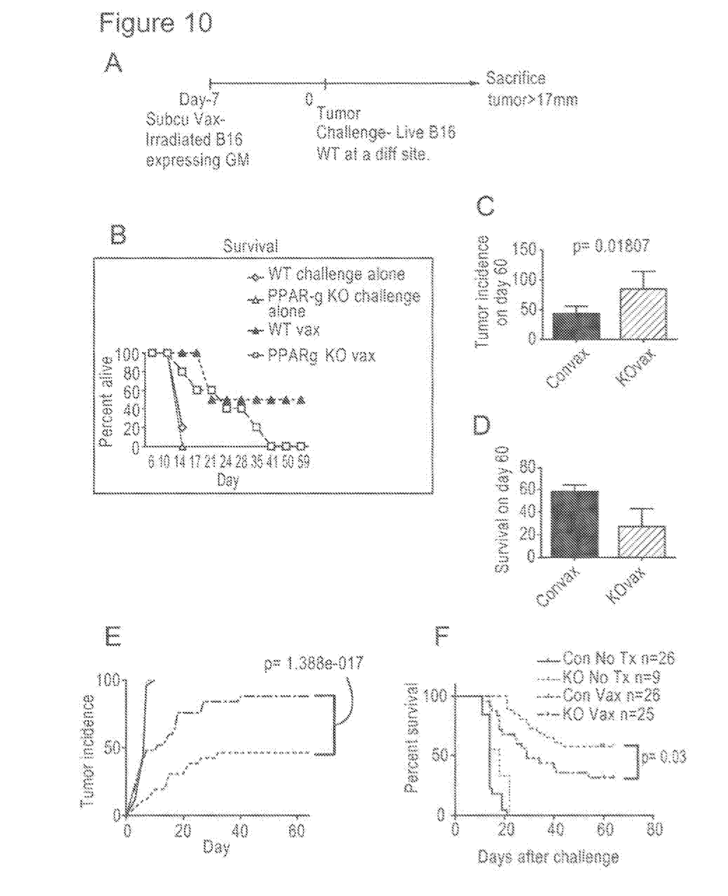

Tissue specific deletion of PPAR-γ using LysM-Cre mice allowed us to address the role of myeloid PPAR-γ in GVAX induced anti-tumor immune responses. Mice prophylactically given GVAX (a week before challenge with live WT tumor cells) were protected from tumor growth and showed long term tumor free survival (FIG. 10a, b). Tumor growth was not impacted by loss of myeloid PPAR-γ in the absence of prior vaccination (FIG. 10b). Surprisingly, we found that vaccine efficacy was reduced in PPAR-γ KO mice (FIG. 10b—representative survival curves, statistics on (c), tumor incidence and (d), survival, from four repeats of the study).

These data indicate that PPAR-γ function in LysM-Cre expressing cells is required for full GVAX induced protective immunity. This is an unexpected finding given the immunosuppressive roles that PPAR-γ is known to play. We sought candidate mechanisms to explain the loss of vaccine efficacy in the PPAR-γ KO and found reports that suggested that PPAR-γ function in DC might be important for optimal NKT cell activation. We know from CD1d KO mice that NKT cells are required for GVAX induced tumor protection. In our studies in CD1d KO mice, loss of NKT cells resulted in the loss of GVAX induced Th2 responses, as measured with splenocytes that were restimulated in vitro with irradiated B16 cells [5]. This GM-CSF/NKT cell/Th2 cytokine axis might be relevant to the loss of vaccination activity in the PPAR-γ deficient mice, as PPAR-γ has been postulated to be important for “M2” activation of macrophages. PPAR-γ KO mice on the Balb/c background are deficient in the Th2 response to Leishmania [6]. Thus, using the restimulation assay as well as other ex-vivo and in vitro assays we tested if NKT function or Th2 generation was impaired in PPAR-γ KO mice.

Restimulation of splenocytes from vaccinated mice does not identify any obvious defect in the PPAR-γ KO anti-tumor cytokine response

Splenocytes harvested a few days after vaccination and cultured with irradiated B16 show a marked cytokine response. Our laboratory has previously shown that the Th2 component of this response is NKT cell mediated. We found that PPAR-γ KO mice generated comparable or slightly enhanced levels of the Th1 and Th2 type cytokines tested, which included IFN-γ, GM-CSF, IL-5, IL-13, and IL-10 (Table 1).

TABLE 1
PPAR-γ KO splenocytes restimulated with B16 cells do not show
an alteration in their cytokine profile.

| Cytokine | Con | KO | Con | KO |
|---|---|---|---|---|
| (ng/ml) | Naïve + irB16 | Naïve + irB16 | Vax + ir B16 | Vax + ir B16 |
| IFN-g | 1.3-8.3 | 1.7-3.9 | 3.6-18.3 | 5.2-18.3 |
| GM-CSF | ND | ND | 0.1-1.0 | 0.3-0.8 |
| IL-5 | ND | ND | 0.9-3.7 | 1.9-6.4 |
| IL-13 | ND | ND | 1.8-6.2 | 3.5-9.9 |
| IL-10 | ND | ND | 0.5-6.3 | 1.5-7.4 |

Data are representative of 5-6 mice. 2 × 10\hat{ }6 splenocytes were cultured with 50,000 irradiated B16 cells. Cytokine levels in the supernatants were measured by ELISA.
ND—not detected.

CD1d Expression is Unaffected in PPAR-γ KO Mice

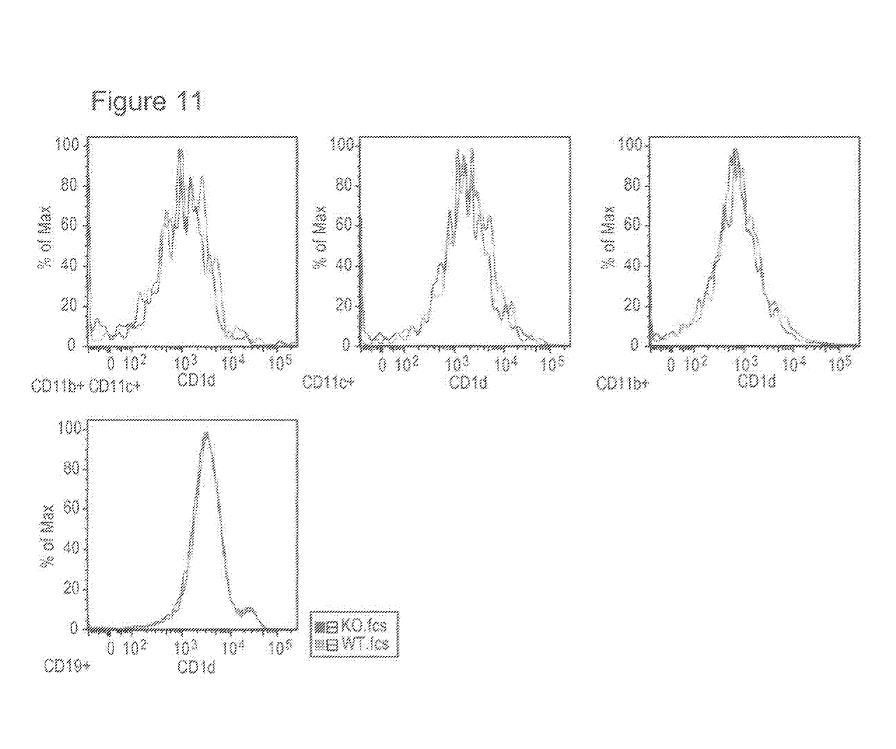

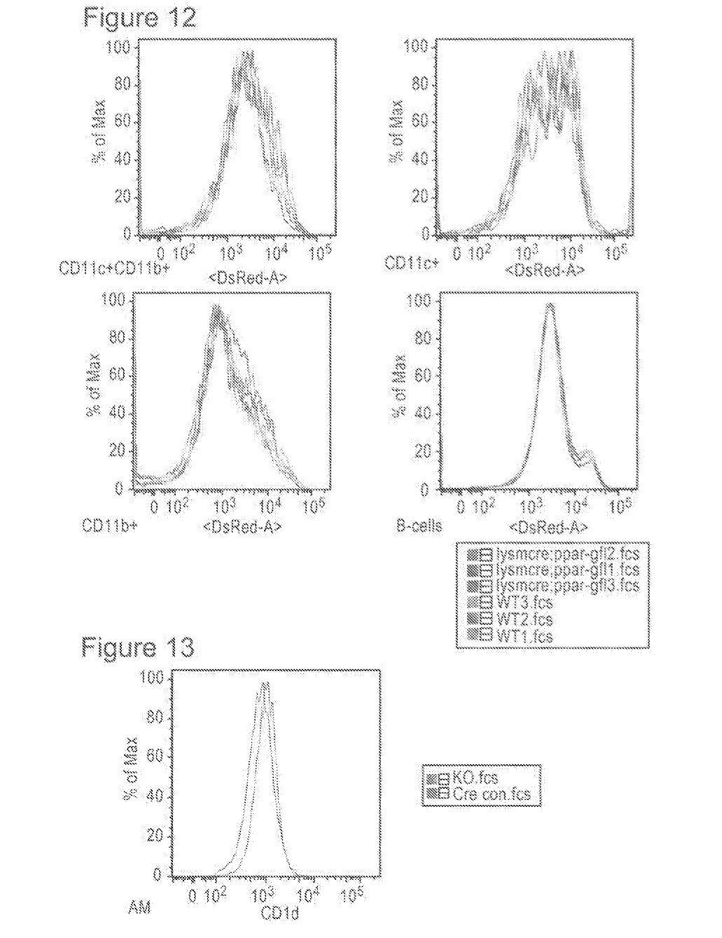

The PPAR-γ ligand Rosi has been shown to induce CD1d expression in GM-CSF and IL-4 derived human DC. This increased expression leads to an increase in NKT cell activation [7]. Thus we tested the expression of CD1d in PPAR-γ KO myeloid subsets. As shown in FIG. 11, we did not detect a difference in CD1d expression in naïve splenic myeloid subsets defined by either CD11b or CD11c expression. CD11b+CD11c− cells can either be monocytes, macrophages or neutrophils. CD11b+CD11c+ cells are considered to be monocyte derived dendritic cells where CD11c+CD11b− cells are classical DC. Further classification is possible using numerous available markers but we used these two markers as a tool to do a preliminary screen of CD1d expression. We found that in a variety of myeloid cell populations in the naïve spleen, CD1d expression was unaffected in the PPAR-γ KO. As a control, we also tested B-cells, a subpopulation of which (marginal zone B cells) is known to express high levels of CD1d, and found that both wild type and PPAR-γ deficient cells showed comparable expression. However, from studies described in chapter 2 and later confirmed by Gautier et al [8], we knew that PPAR-γ is not easily detectable in myeloid splenic subset in steady state. We tested splenic myeloid cells in vaccinated mice and again found no defect in the PPAR-γ KO (FIG. 12). As PPAR-γ was robustly detectable in alveolar macrophages, we tested if expression in alveolar macrophages was affected. Even alveolar macrophages from PPAR-γ KO mice had no defect in CD1d expression (FIG. 13).

Flow Cytometry Did not Reveal a Major Defect in the PPAR-γ KO APC from Live B16-GM Vaccination Sites

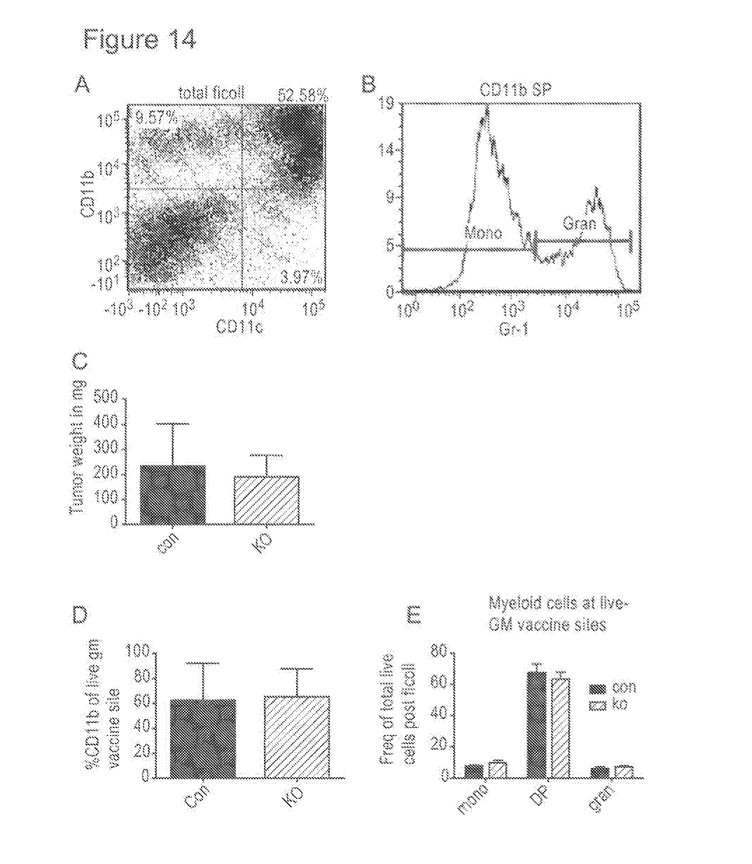

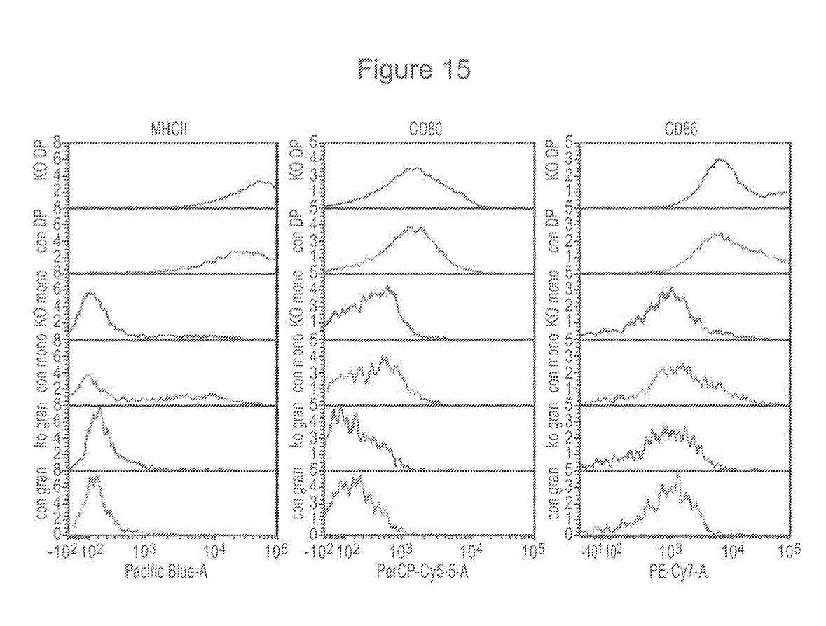

We again used CD11b and CD11c as markers to identify myeloid population in the live-GM vaccine sites (FIG. 14a). In addition, we used Gr-1 to categorize CD11b single positive cells as granulocytic (Gr-1+) or monocytic (Gr-1-) (FIG. 14b). As these vaccine sites are in fact, progressive tumors, it is important to note that their growth was not affected in the PPAR-γ KO mice (FIG. 14c). Neither did we did not find any difference in the frequencies of various myeloid subsets as defined by CD11b, CD11c and Gr-1 in the PPAR-γ KO vaccine sites (14d and e) or their absolute numbers (data not shown). We further tested the activation status of the granulocytic, monocytic and dendritic fraction by using MHCII, CD80, and CD86 and did not find any difference in the PPAR-γ KO (FIG. 15).

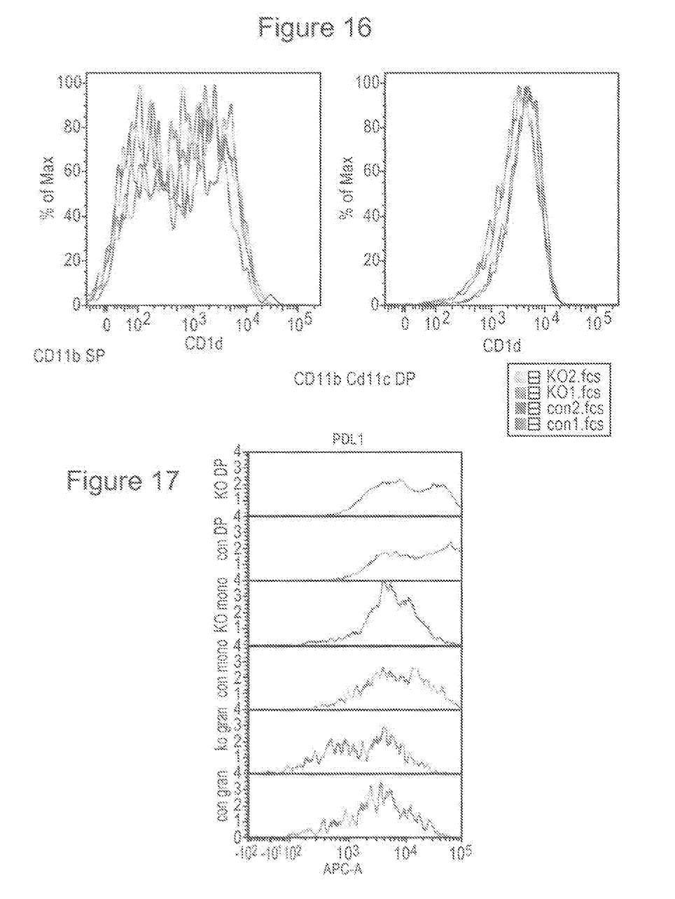

We then tested the CD11b SP (monocytes and granulocytes) and CD11c CD11b DP (monocyte derived dendritic cells) for their expression of CD1d. As shown in FIG. 16, we found no defect in CD1d expression in vaccine site APC from PPAR-γ KO mice. Thus, we concluded that PPAR-γ loss in LysM expressing cells does not affect murine CD1d expression. We wondered if the published data reporting Rosi effects on cultured human DC could be used to reveal more candidates that might contribute to the reduced vaccine efficacy in the PPAR-γ KO. We reanalyzed publically available datasets and found that PD-L1 expression is reduced by Rosi treatment of cultured human DC (bioinformatics analysis performed by Vladimir Brusic and David Deluca at the DFCI Bioinformatics Core). This suggested that PD-L1 expression could be upregulated in the PPAR-γ KO. PD-L1 is known to be induced by GM-CSF and increased expression on APC could lead to reduction in vaccine efficacy. However, flow cytometric analysis of cells recruited to the vaccine site did not show any defect in PD-L1 (FIG. 17).

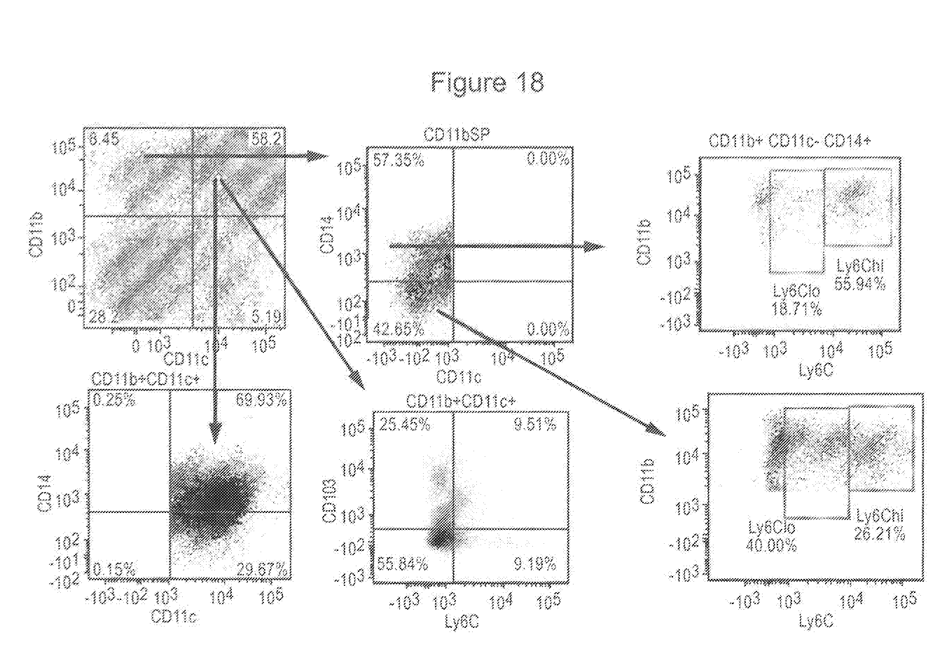

The wide range of expression of these activation markers suggested that further sub categorization of these myeloid cells would be possible. We tested the markers CD14, CD103 and Ly6c (FIG. 18). CD14 and Ly6c expression are seen on monocytes. Ly6c expressing cells can be further subdivided into Ly6hi (inflammatory monocytes) and Ly6lo. CD103+DC are found in several anatomical sites and maintain tolerance via Treg under homeostatic conditions. Yet they are very efficient at cross presentation and mounting a CD8 response during an immune response [7]. GM-CSF KO have reduced numbers of CD103+DC in several non-lymphoid compartments. We did not detect a difference in any of these markers or subpopulations in the PPAR-γ KO (data not shown).

Coculture of Vaccine Site APC with Various Effector Cell Subsets Did not Reveal any Defects in the PPAR-γ KO

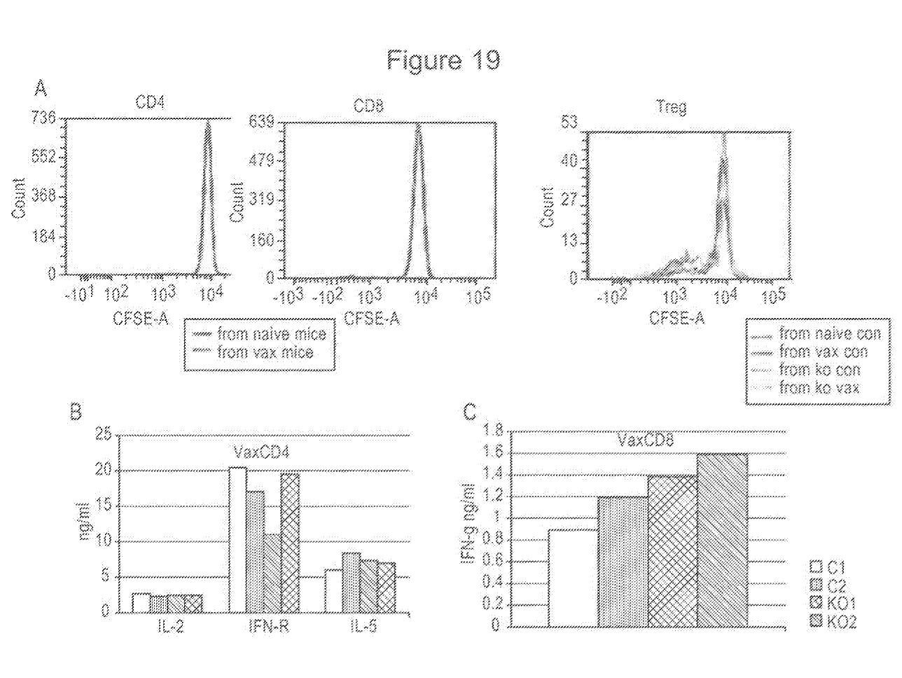

Based on expression markers, it appeared that PPAR-γ KO APC from vaccine sites were present and expressed similar surface markers compared to wild type mice. We extended our analysis to investigate the functional capacity of the PPAR-γ deficient APCs. We cultured myeloid cells (using CD11b and CD11c as markers) from B16-GM tumors with CD4 and CD8 cells from the spleens of naive or vaccinated mice. The only T-cells which proliferated in these assays in response to the vaccine site APC were FoxP3+CD4+ regulatory T-cells from naive mice (FIG. 19a). GM-CSF is known to be required for Treg homeostasis in the gut and can promote Treg in culture. There was no difference in Treg proliferation when the Treg were cultured with vaccine site PPAR-γ KO APC as compared to control APC (FIG. 19a). CD4 and CD8 from vaccinated mice produced cytokine in response to the APC but the levels of IL-2, IFN-γ and IL-5 production by CD4 (19b) and IFN-γ production by CD8 (19c) were not different if the APC were derived from PPAR-γ KO mice.

We also continued our investigation into the role of NKT cells, if any, in the vaccine defect in the KO. In one study, lipid antigen availability on CD1d was suggested to be modulated by PPAR-γ induced cathepsin D Thus we cultured NKT with vaccine site APC to measure their cytokine responses. Two different sources of NKT cells were used: cell lines or primary NKT cells derived from Vb7 restricted mice generated by somatic cell nuclear transfer (Stephanie Dougan, unpublished data). Briefly, a nucleus from a Vb7 expressing NKT cell was extracted and placed in an enucleated oocyte which was then allowed to grow to the blastocyst stage. Embryonic stem cell lines derived from the Vb7 blastocyts were injected into WT blastocysts. Chimeric blastocyst were implanted in pseudopregnant mice. The resulting chimeric pups can be mated to obtain Vb7 mouse lines. Since the TCRa locus does not display absolute allelic exclusion in WT animals (30% of all T cells have both alleles of TCRa rearranged and 10% express both alleles), the T cell compartment in extremely restricted but not clonal in these mice. The T-cell compartment in the Vb7 mice is skewed towards NKT cell development though some CD8 T cells are present.

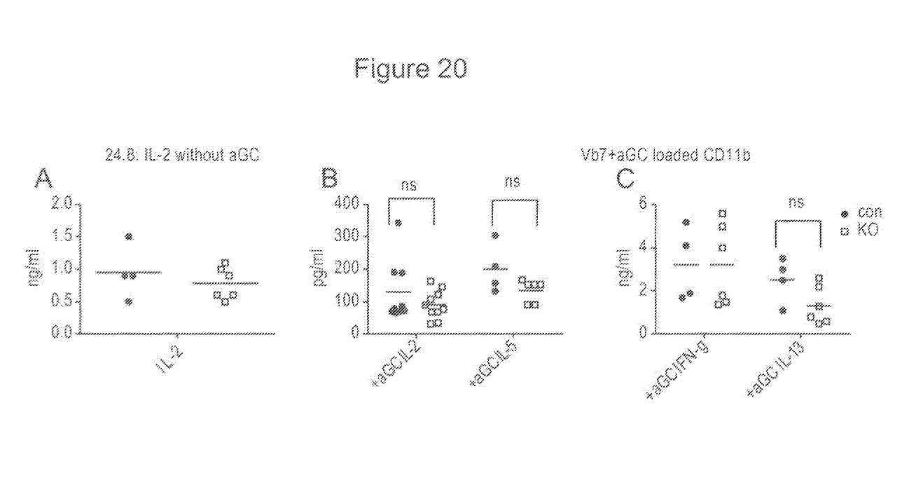

Cytokine profile of a NKT cell line (24.8) or primary Vb7 NKT cells was similar in the presence of APC from con or KO vaccine sites (FIG. 20). The only cytokine detectable on coculture of CD11b+ cells from live-GM vaccine sites and 24.8 cells was IL-2, which was not markedly affected by loading the CD11b cells with α-galactosylceramide (aGC, data not shown). There was no difference in IL-2 production by 24.8 cells when stimulated with KO APC (FIG. 20a). Primary Vb7 NKT cells produced IL-2, IL-5 (FIG. 20b), IL-13 and IFN-γ (FIG. 20c) on aGC stimulation but not with endogenous ligands. Any alteration in CD1d expression or recycling in the PPAR-γ KO APC would impact the NKT cell response to aGC. Similarly if costimulatory ligands, either cell surface or secreted, differ in the KO, it may impact NKT cell response to aGC. However, the cytokine response of primary NKT cells to aGC loaded APC also remained unchanged when PPAR-γ KO APC were used

### Discussion

PPAR-γ is known to have many immunosuppressive functions in macrophages and dendritic cells. Contrary to our expectation, deletion of PPAR-γ using LysM-Cre reduced the ability of irradiated, GM-CSF secreting B16 cells to stimulate protective immunity against subsequent tumor challenge. Although prior reports suggested a role for PPAR-γ in NKT cell activation, we failed to detect a clear defect involving NKT cells in the PPAR-γ deficient mice. Instead, we found that a) CD1d expression was unaffected in PPAR-γ KO mice and b) NKT cell activation by vaccine site APC as measured by cytokine release was also unaffected. We also conducted coculture assays with the vaccine site APC with CD4 and CD8 cells from naïve and vaccinated mice but were unable to reveal a defect in the PPAR-γ KO vaccine sites. Moreover, similar myeloid cells were recruited to the site of GM-CSF secreting tumor cells in wild type and PPAR-γ deficient mice. Together, these results raised the possibility that a previously unknown function of PPAR-γ might be involved in the impaired vaccination response, an issue that we address with detailed expression profiling analysis in the next example.

### Example 4: High Throughput Analysis of Gene Expression in GVAX Draining Lymph Node and Identification of a Novel Role of PPAR-r in Myeloid Cells

Methods

RNASeq

dLN were harvested 5 days after vaccination. LN from 4 mice were pooled and RNA was extracted. RNA was subjected to HiSeq and transcript levels determined for approximately 20,000 genes (Center for Canter Computational Biology, DFCI). 2 technical repeats were performed for con and 3 for KO.

Gene Set Enrichment Analysis (GSEA)

GSEA was performed using all available genesets in the Immgen database (˜300 at the time) to identify modules and associated cell types whose gene signature were differentially represented in con or KO LN.

Combinatorial Immunotherapy

For the experiments exploring synergy of GVAX+CTLA-4 with Rosi, we used two different challenge doses: 10̂5 or 4×10̂5. Vaccination dose was 3×10̂6 cells B16-GM, injected once, subcu. on the abdomen, opposite to the flank with the challenge dose. Rosi or DMSO were given in drinking water at 20 mg/kg/day for 12 days. Mice were injected i.p. with anti-CTLA-4 (9D9, BioXcell) or isotype as follows: 200 ug on d0, 100 ug on d3 and d6.

Results

Gene Expression Profiling of Vaccine Draining Lymph Node

In support of the protective response induced by the vaccine, ipsilateral inguinal lymph nodes were dramatically enlarged morphologically and in cellularity (5-10 fold, data not shown). To analyze the vaccine effector mechanisms without bias towards one particular cell type, we collected RNA from draining lymph nodes 5 days after vaccination and conducted RNA-Seq. Draining lymph nodes from 4 mice were pooled to reduce variability.

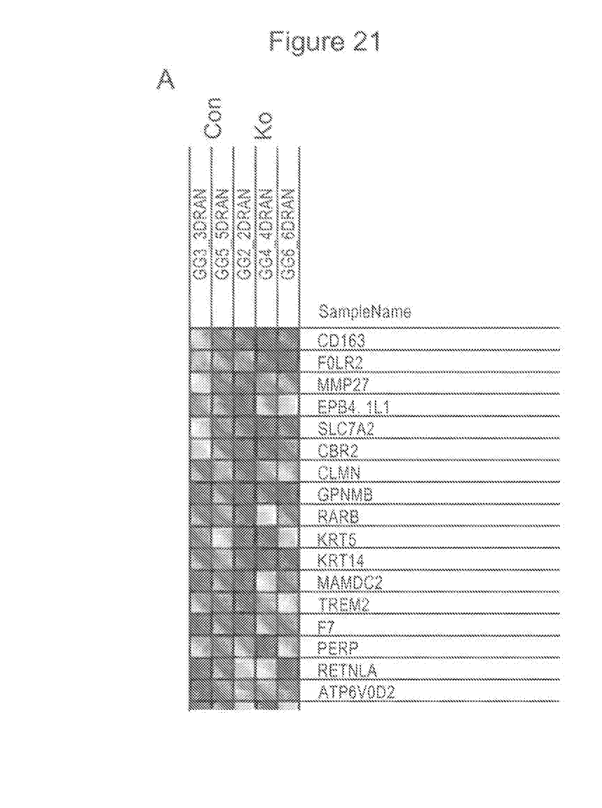

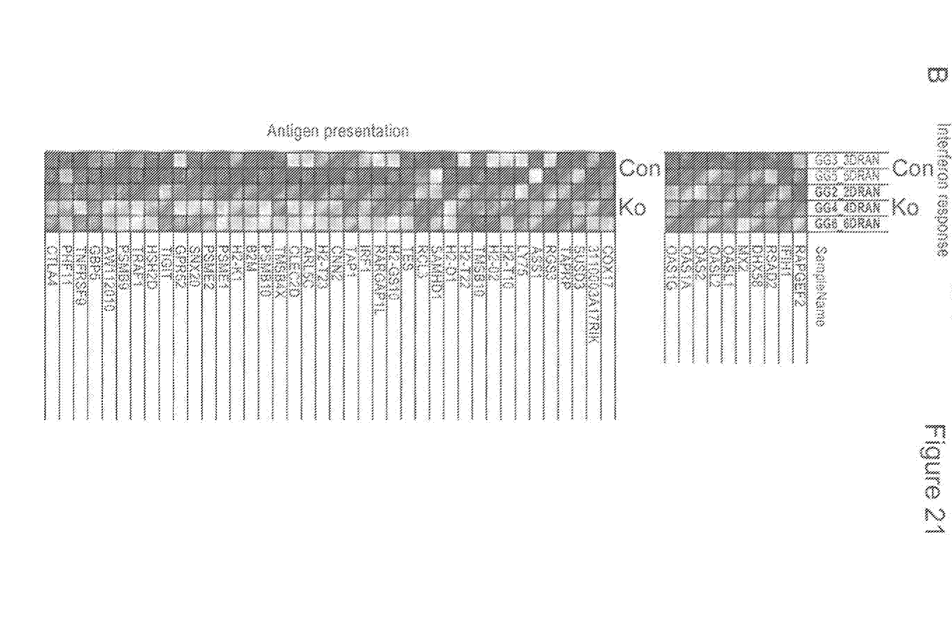

To identify changes in gene expression in the draining lymph node, the transcript levels obtained from the RNASeq data were analyzed by gene set enrichment analysis (GSEA). We used the genesets available through Immunological Genome project consortium (Immgen.org) to identify modules corresponding to specific cell types and signaling pathways. Interestingly, a PPAR-γ dependent gene expression module previously shown to be enriched in alveolar macrophages was underrepresented in the PPAR-γ KO lymph node compared to controls, confirming that PPAR-γ dependent myeloid gene expression was reduced (FIG. 21a). Genesets that are known to be repressed by PPAR-γ were upregulated in the KO confirming functional deficiency of PPAR-γ (FIG. 21b). Together, these findings indicated that more detailed analysis of the gene expression profiles might provide insights into the impaired vaccine responses in PPAR-γ deficient mice.

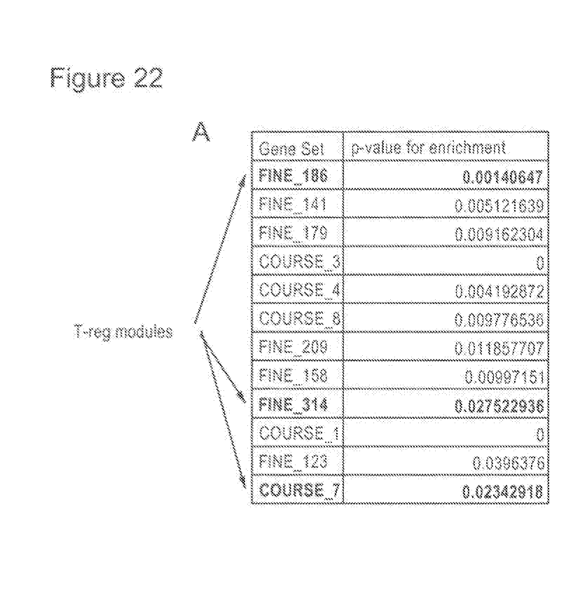

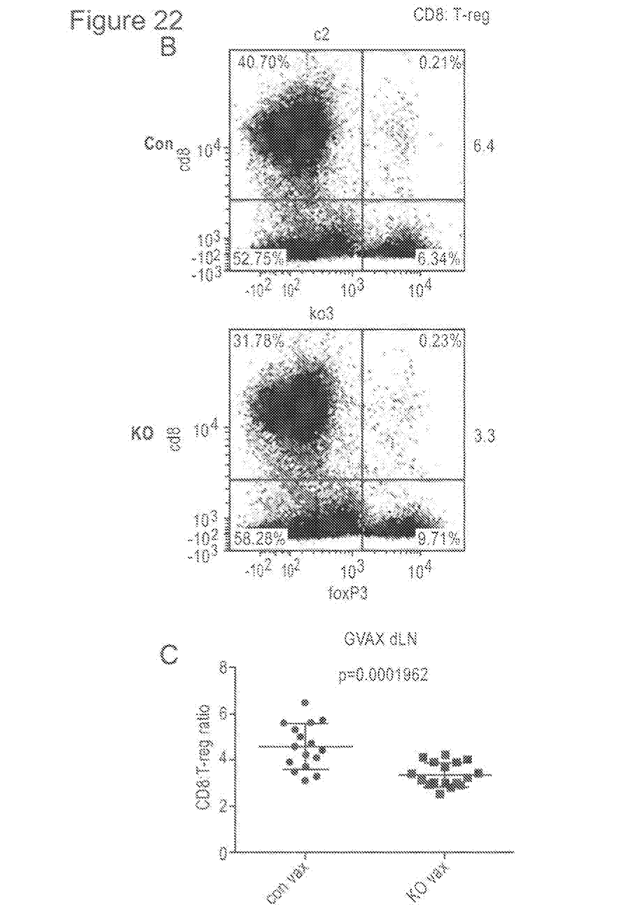

Interestingly, CTLA4 was one of the top genes showing upregulation in KO lymph nodes (last gene FIG. 21b, previous page). CTLA4 is strongly expressed on regulatory T-cells and on activated and exhausted effector cells. GSEA showed gene expression modules specific to Treg are upregulated in the KO (FIG. 22a.). We sought to confirm this possible alteration in Treg by flow cytometry. As shown in FIG. 22b, Treg frequency is increased. As Treg are a major regulator of anti-tumor effector T cells, we wondered whether this might impact CD8+ T cells. Indeed, the CD8:Treg ratio was decreased in KO draining lymph nodes compared to control mice 6-8 days after vaccine administration (FIG. 22c).

Interestingly, CTLA4 was one of the top genes showing upregulation in KO lymph nodes (last gene FIG. 21b, previous page). CTLA4 is strongly expressed on regulatory T-cells and on activated and exhausted effector cells. GSEA showed gene expression modules specific to Treg are upregulated in the KO (FIG. 22a.). We sought to confirm this possible alteration in Treg by flow cytometry. As shown in FIG. 22b, Treg frequency is increased. As Treg are a major regulator of anti-tumor effector T cells, we wondered whether this might impact CD8+ T cells. Indeed, the CD8:Treg ratio was decreased in KO draining lymph nodes compared to control mice 6-8 days after vaccine administration (FIG. 22c).

CD8:Treg Ratio in the Tumor is Also Reduced in the KO

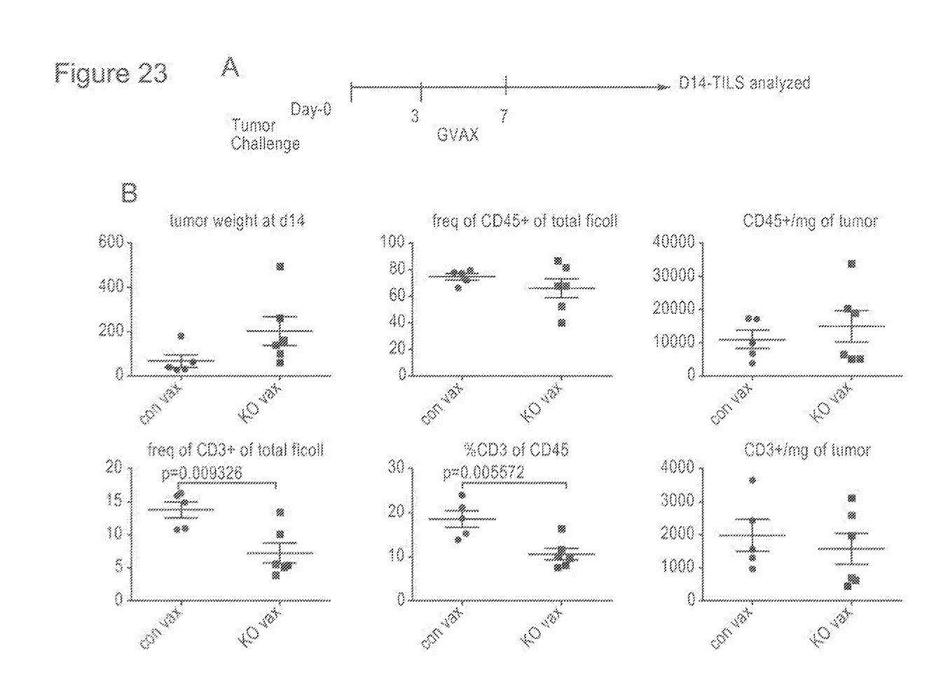

We wondered if the altered balance of CD8 T effectors and FoxP3+ Treg observed in the draining lymph nodes were also seen at the tumor site. For these experiments, we moved to a therapeutic vaccine model such that all mice would have progressive tumors. As shown in FIG. 23, d14 B16 tumors from GVAX treated KO mice did not show an alteration in the frequency of CD45+ cells in single cell suspensions of tumors. However, the frequency of CD3+ T cells was significantly reduced in the KO mice compared to controls. There was a non-significant trend towards larger tumor sizes. The lack of effect on tumor growth might reflect the limited ability of GM-CSF secreting tumor cell vaccines to impact the progression of established tumors, precluding the ability to detect a major PPAR-γ dependent contribution. Similar results were obtained with dll tumors (data not shown).

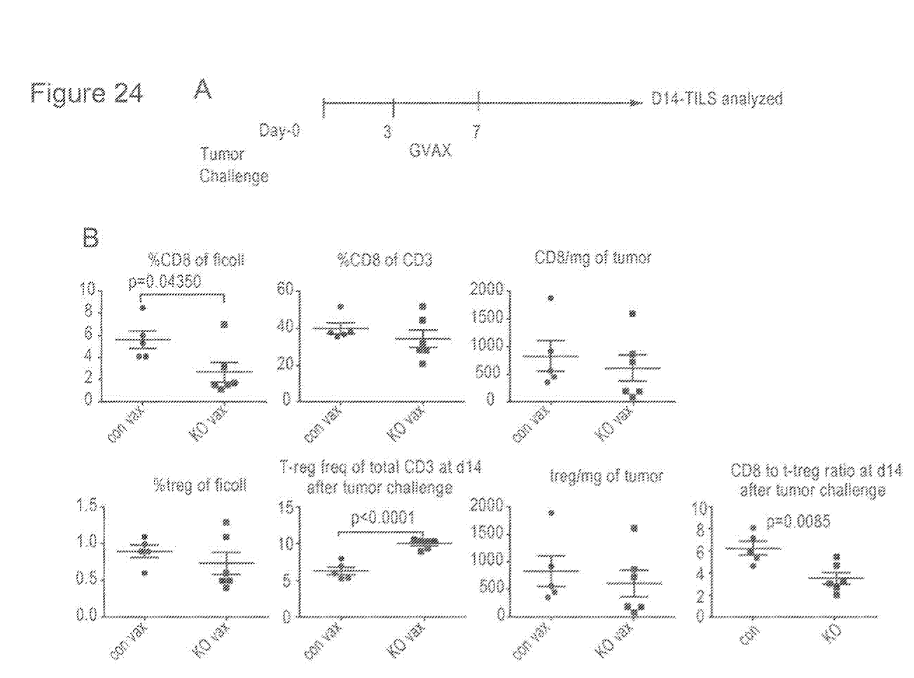

We further categorized the CD3 infiltrate based on CD8, CD4 surface expression and intracellular staining for FoxP3. Total recovery of CD8 and Treg was reduced in the KO tumors as expected due to lower CD3 (FIG. 24). However, this effect was more pronounced (and statistically significant) in the CD8 compartment, leading to lower CD8:Treg ratios in the KO mice. The balance of CD8 to Treg at the tumor site has emerged as an important prognostic variable for a number of cancers in clinical studies.

Impact on DC Associated Genes in KO dLN and T-Cell Stimulation by DC

To identify the origin of the reduced CD8:Treg ratio we analyzed the draining LN. We performed high throughput and unbiased analysis of whole LN by RNASeq. The draining LN from each mouse was collected 5 days after GVAX administration. 4-5 draining lymph node were pooled for each sample.

Myeloid cells are a relatively rare population in the draining lymph node. So, we first tested if deletion of PPAR-g using LysMCre leads to an identifiable difference in PPAR-g target gene expression in the RNASeq of the whole draining LN. We found that several canonical PPAR-g target genes were reduced in KO draining lymph node (FIG. 38A). Previously, a PPAR-g controlled gene module has been identified in alveolar macrophages. Several of the macrophage genes in this module were also reduced in the KO (FIG. 38B). These data confirmed that we were able to identify myeloid restricted gene expression changes in our whole lymph node RNASeq and also validated a functional defect in PPAR-g.

We then took all gene modules (˜300) profiled in the Immgen database (Immgen.org, a consortium of laboratories profiling all immune cell types in the mouse by microarray). Gene modules are defined as genes that were found to be coexpressed and are annonated with the cell type and stimuli in which they are expressed. We asked by gene set enrichment analysis (GSEA) if any of these gene modules were changed in the KO. We found that a large geneset enriched in dendritic cells was reduced in the KO (FIG. 38C, coarse module 26 on Immgen.org). The DC enrichment in empirically defined by microarray analysis of various murine immune cell subsets on Immgen.org

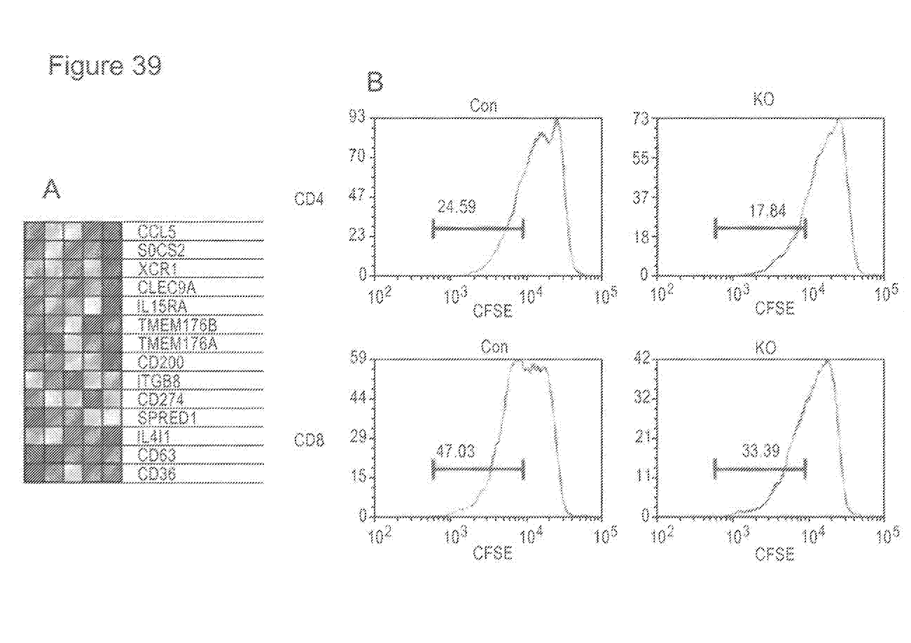

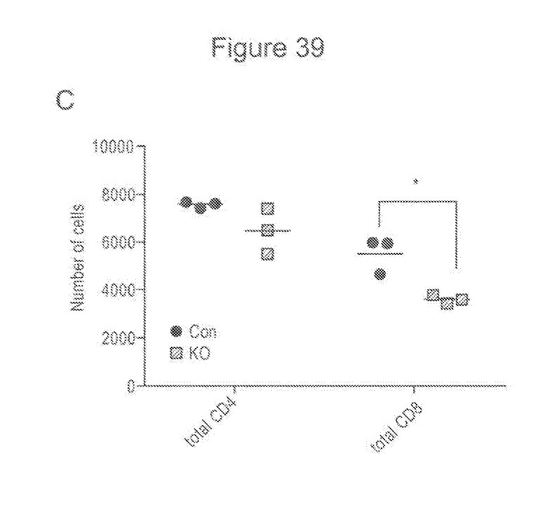

Flow cytometric analyses showed that a large fraction of the CD11c+(a marker for DC) cells in the draining lymph node were MHCII hi and CCR7+ suggesting that the major DC population in the GVAX draining lymph node were migratory DC (data not shown). Previous studies have identified a signature of tolerance in naïve murine migratory DC. We found the naïve tolerogenic signature of migDC were retained in the KO dLN (FIG. 39A) in contrast to the many DC genes that were downregulated. These data suggested reduced immunostimulatory potential of dendritic cells in the KO draining lymph node. In mixed lymphocyte reactions (MLR), CD11c+ cells from KO dLN had a reduced capacity to activate T cells (FIG. 39B, 39C).

Impact on Regulatory T Cell Gene Signature and CD8:Treg Ratio in KO dLN

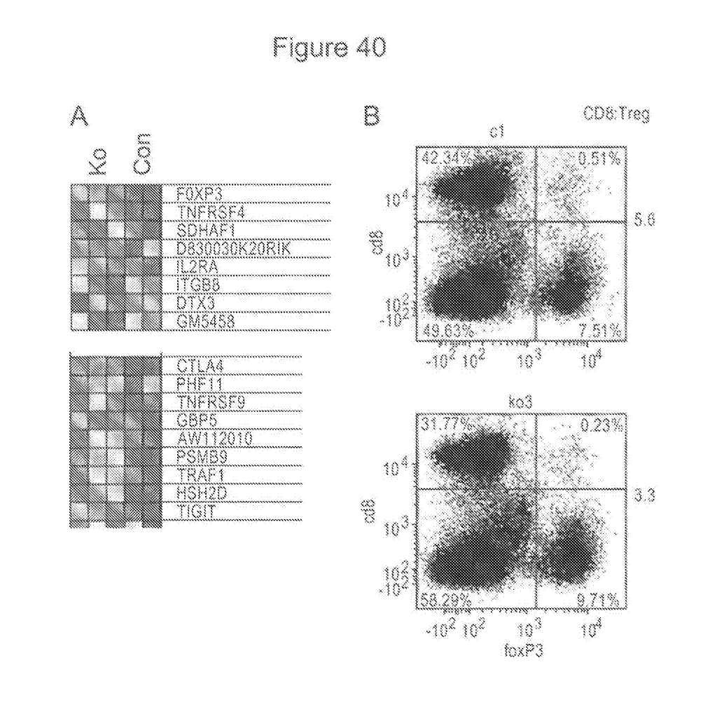

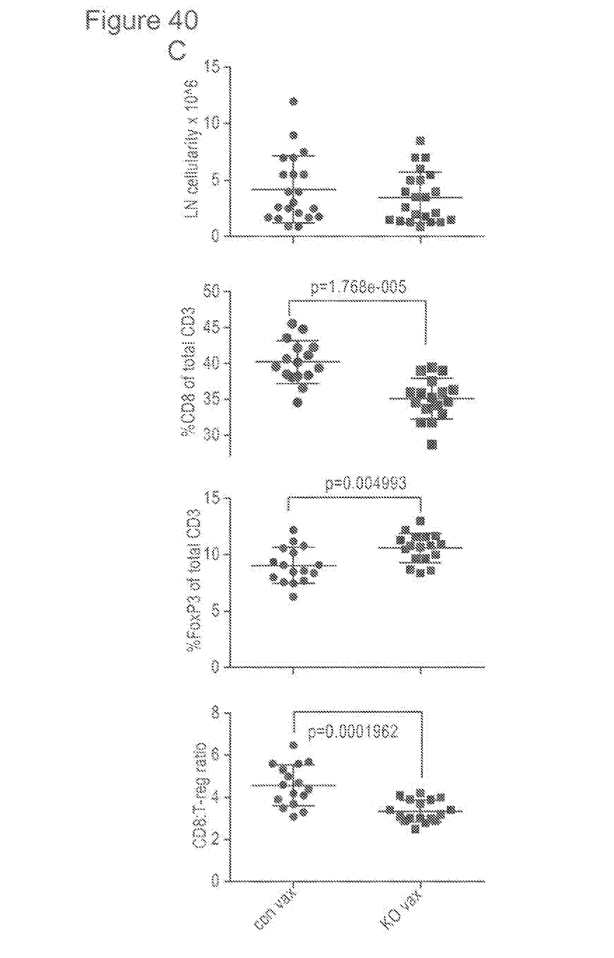

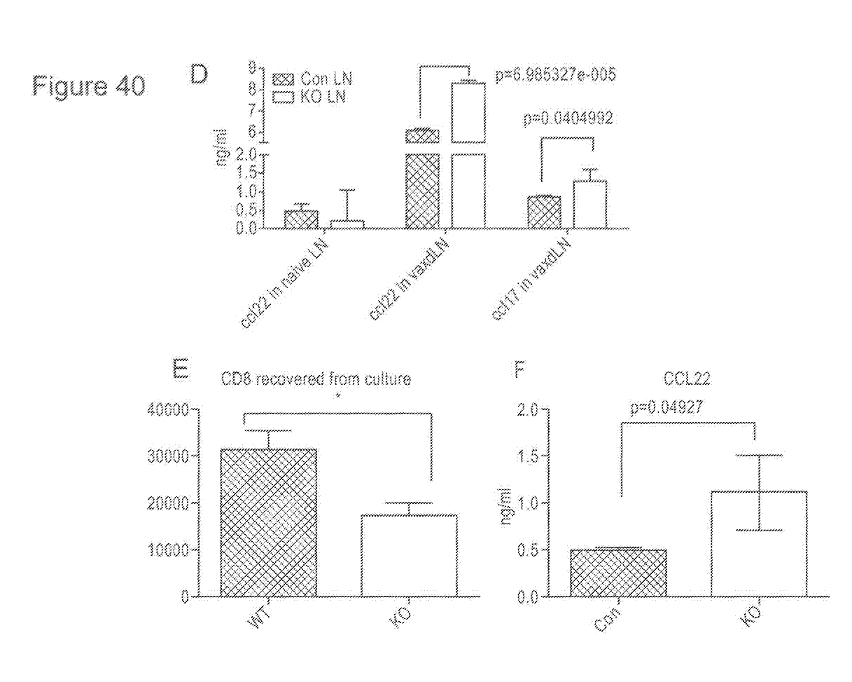

The DC gene signature and functional defects suggested an impact on T cell function. Thus, we next assessed if the RNASeq revealed any T-cell defects. In accordance with the increased Treg at the tumor site, we found an increase in the Treg specific gene modules in the KO (FIG. 40A). Treg specific or enriched genes such as FoxP3, IL2RA (CD25), CTLA-4 were significantly enriched in KO dLN. We confirmed an increase in Treg frequency by flow cytometry (FIGS. 40B and 40C). Interestingly, in accordance with the TIL data and reduced T cell proliferation in MLR, we also found a decreased frequency of CD8 and a reduced CD8:Treg ratio (FIG. 40C) in KO dLN.

To explore the basis of increased regulatory T cell frequency, we again focused on the dendritic cell gene expression. In KO dLN, we found an increased expression of CCL22 (data not shown), a chemokine produced by myeloid cells, known to recruit regulatory T cells. A similar function is performed by CCL17 which shares a common receptor with CCL22. Thus we tested the expression of CCL17 and CCL22 by ELISA in con and KO dLN. We found increased levels of both chemokines in KO dLN providing a possible link between PPAR-g deficiency in the draining LN DC and the impact on Treg (FIG. 40D).

To understand if these effect of the tissue specific deletion of PPAR-g extended to our cutaneous vaccination models, we compared the level of CCL22 and CD8 survival in dLN from con and KO mice scarred with vaccinia virus. We found that CD8 survival was decreased (FIG. 40E) and CCL22 was increased (FIG. 40F) in KO mice compared to con mice in the vaccinia scarification model also. These data suggest a requirement for myeloid PPAR-g in promoting protective T cell function in cutaneous vaccination.

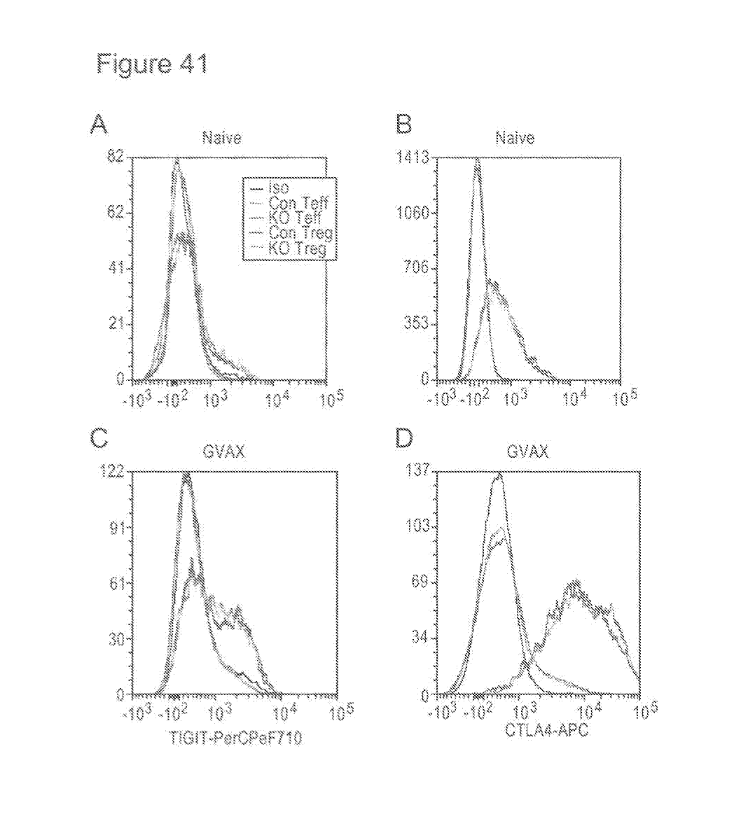

We were interested to note that the expression of coinhibitory receptors CTLA-4 and TIGIT was upregulated in the KO (FIG. 40A). We found limited cell surface expression of these receptors in the naïve LN (FIG. 41A, B). In GVAX treated mice, the major T cell population expressing these receptor in dLN was the FoxP3 positive regulatory subset (FIG. 41C, D). FoxP3− cells, collectively labeled as “effector T cells) remained CTLA-4 and TIGIT negative even after vaccination (FIG. 41C, D). Further, we found that all intratumoral Treg in GVAX treated mice (in con and KO) were TIGIT positive.

Pharmacological Activation of PPAR-g by Rosiglitazone Improves the Response to Immunotherapy

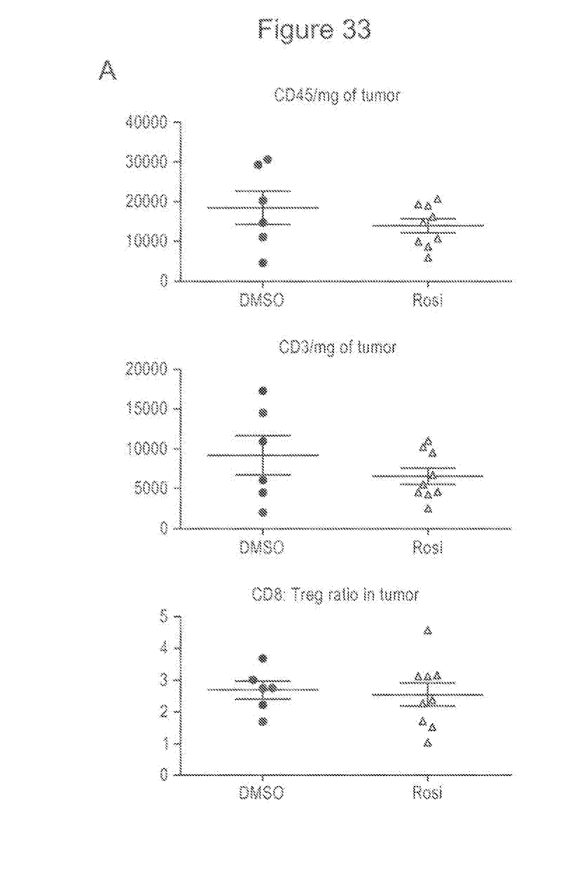

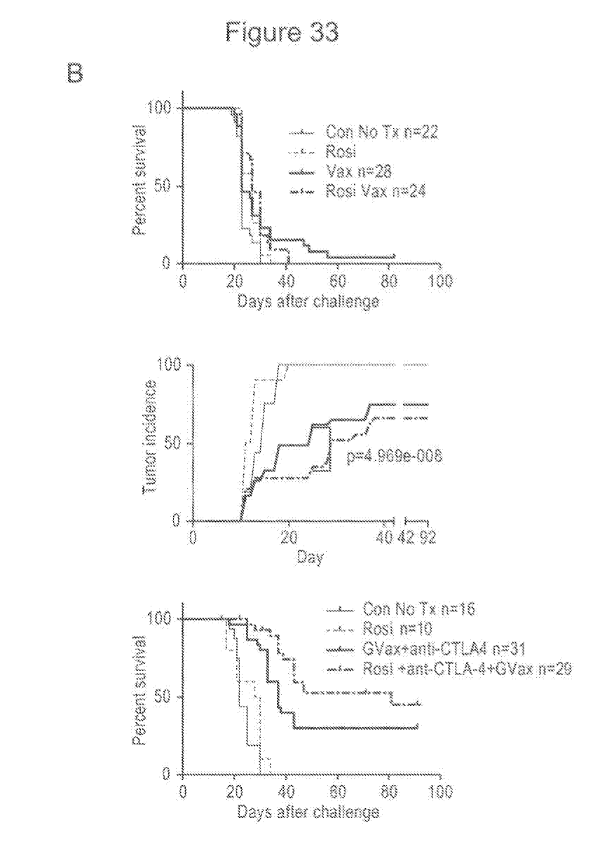

In the previous experiments, we found that a genetic deficiency in PPAR-g in Lysozyme M expressing cells can reduce the efficacy of GVAX. We now queried the whether we could improve the vaccine efficacy by pharmacological gain-of-function. Rosiglitazone is a clinically approved ligand of PPAR-g. We turned to our therapeutic vaccination model to test the intratumoral lymphocytes. We found that in mice treated with GVAX and Rosiglitazone (20 mg/kg in drinking water), the CD8:Treg ratio was increased. The improvement in the CD8:Treg ratio did not occur in the LysM-Cre; PPAR-g fl mice or providing evidence that Rosiglitazone improves the CD8:Treg ratio by acting on myeloid PPAR-g. These data complement the loss-of-function data and provide further evidence that PPAR-g can support the immune response to GVAX. However, despite the improved CD8:Treg ratio, there was no improvement in the survival of the mice (FIG. 33B).

Therapeutic vaccination with GVAX alone is insufficient to show protective efficacy in our regimen (FIG. 33B, top panel). Thus we hypothesized that providing additional immunotherapy in the form of checkpoint blockade might make the model permissive to reveal improvement in survival with rosiglitazone. We used a CTLA-4 blocking antibody, which is a strategy being pursued aggressively used in the clinic. A combination of GVAX and anti-CTLA4 did indeed provide some therapeutic benefit in our model (FIG. 33B, middle and lower panel). Importantly, Rosiglitazone further improved tumor incidence (FIG. 33B, middle panel) and survival (FIG. 33B, middle panel) in GVAX and anti-CTLA4 treated mice.

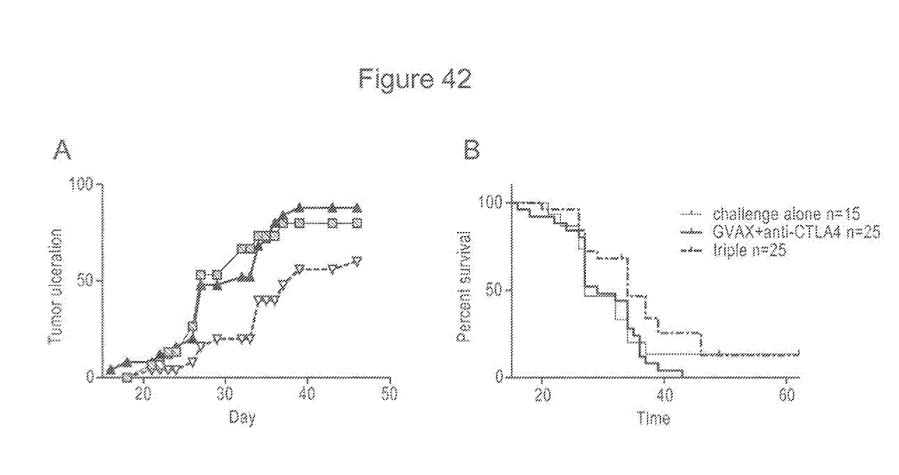

To eliminate the possibility that these data are restricted to one particular cell line, we tested the impact of Rosiglitazone on GVAX+anti-CTLA4 treated mice in another model, a heterotropic lewis lung carcinoma implanted subcutaneously. Lewis Lung Carcinoma is an invasive cell line, which can lead to ulcerations. We found that incidence of ulceration (FIG. 42A) and survival (FIG. 42B) was modestly but significantly improved by Rosiglitazone in GVAX+anti-CTLA-4 treated mice. Thus, our data provide evidence in two independent models, of a novel proinflammatory role of PPAR-g suggesting a new indication of use for the FDA approved drug Rosiglitazone.

Conservation of PPAR-g Function in Human Monocytes

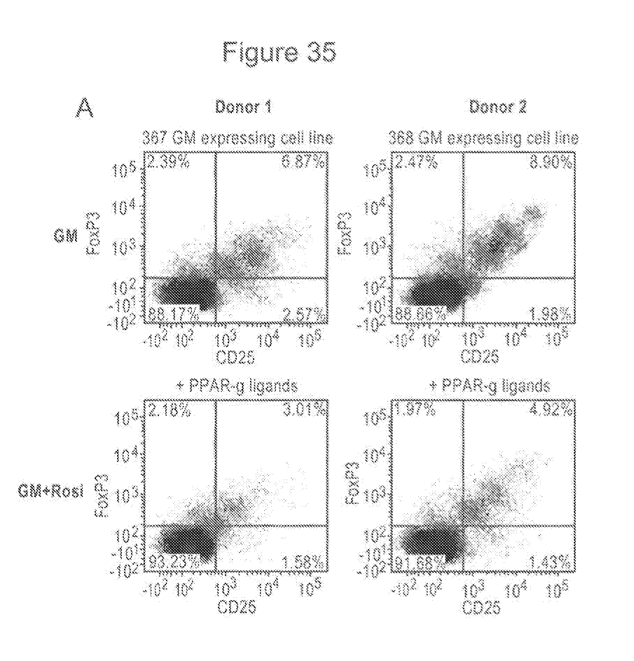

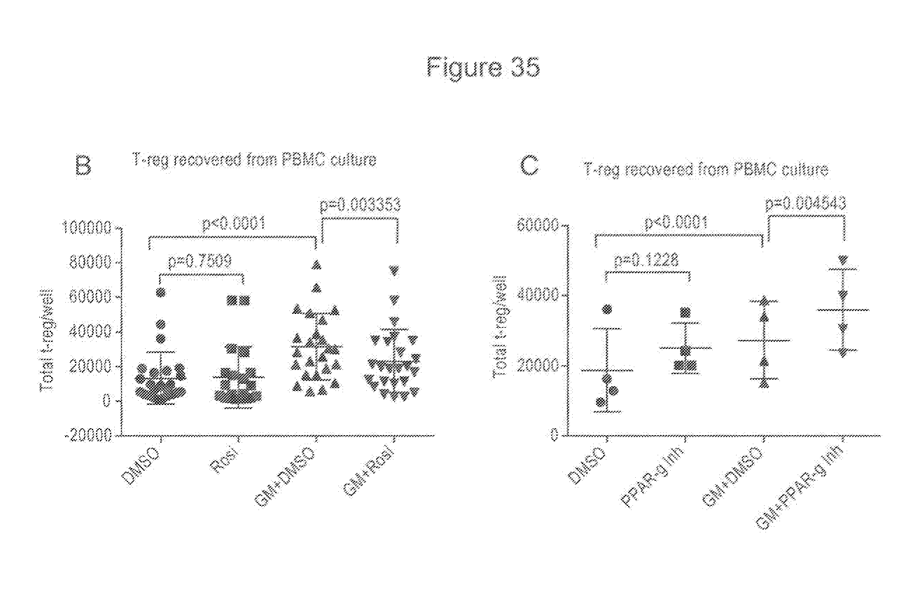

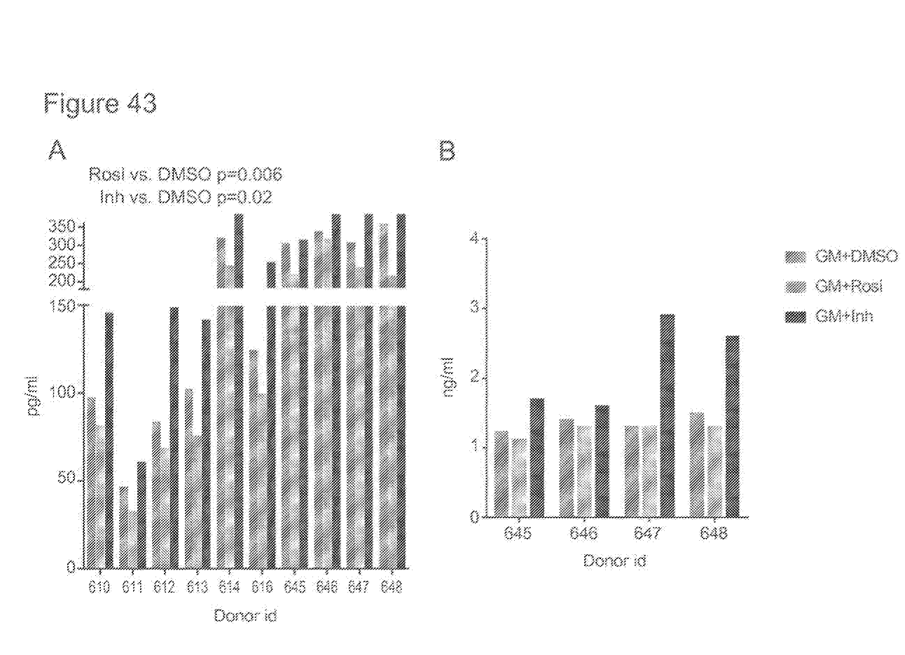

To understand the clinical relevance of these data and to check if this role of PPAR-g is conserved in humans, we treated human monocytes with GM-CSF to induce the expression of PPAR-g. In addition to GM-CSF, the monocytes were treated with Rosi or T0070907, a PPAR-g antagonist. Expression of CCL17 (FIG. 43A) and CCL22 (FIG. 43B) in the monocytes was reduced by Rosiglitazone. Adding to the robustness of these data, treatment with T0070907, gave the opposite effect of increasing CCL17 and CCL22 expression. Further, we found that Rosiglitazone reduced Treg number in these GM-CSF treated monocytes cocultured with autologous T cells, compared to non-GM-CSF treated cultures where it had no effect (FIGS. 35A and 35B).

KO LN have Increased Expression of Treg Promoting Cytokines CCL17 and CCL22

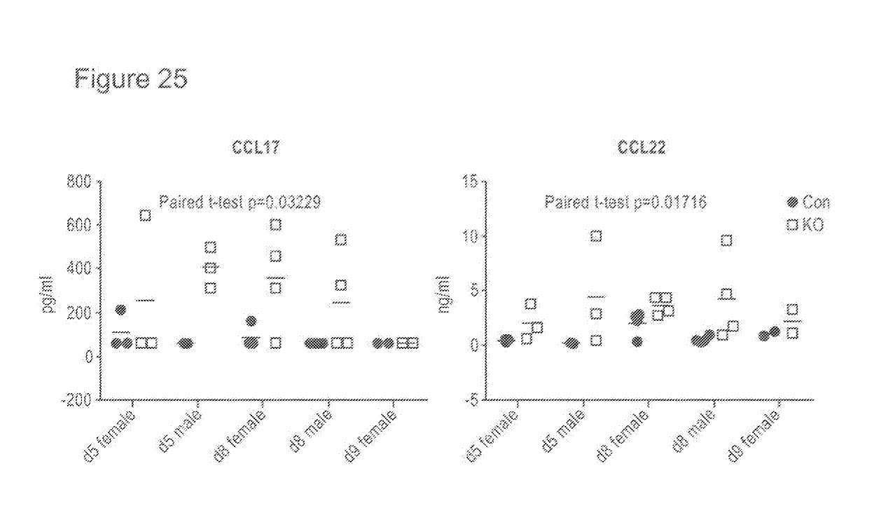

To explain the increased Treg frequency and the effect on the CD8:Treg ratio, we returned to our RNASeq data. Interestingly, the expression of chemokines CCL17 (TARC) and CCL22 (MDC) was upregulated in the KO geneset. CCL17 and CCL22 have been implicated in recruiting Treg via their receptor CCR4. Interestingly, the main subset known to produce CCL17 and CCL22 are macrophages and dendritic cells. We tested the changes in CCL17 and CCL22 production by ELISA. FIG. 25 shows the increased expression of CCL17 and CCL22 by PPAR-γ KO GVAX dLN at 3 different time points.

IFN-γ Response of CD8 T-Cells is not Defective in the KO

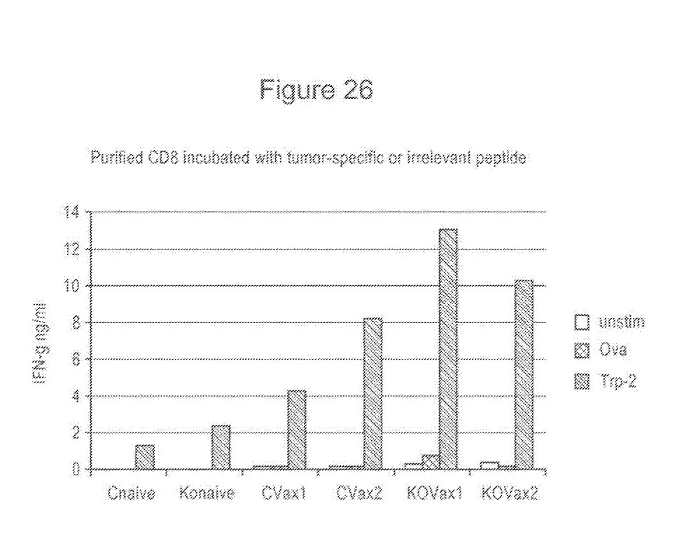

We asked what impact increased Treg had on CD8 function. As shown in FIG. 26, CD8 from con and KO LN secreted equivalent levels of IFN-γ in response to an immunodominant peptide from Trp-2, a melanocyte specific protein that is targeted in the anti-B16 response to GVAX. These data are not surprising as we had already seen equivalent IFN-γ levels in the restimulation of the spleen (chapter 3, Table 2) and the increased IFN-γ response gene signature in the RNASeq (FIG. 21). We are currently optimizing cytotoxicity assays to determine if CD8 mediated killing of B16 tumors is defective as a result of the increased Treg. However, from the RNASeq we did not detect any reduction in granzymes or perforin in the KO (data not shown).

KO LN have an Enhanced Gene Expression Signature for Langerhans Cells

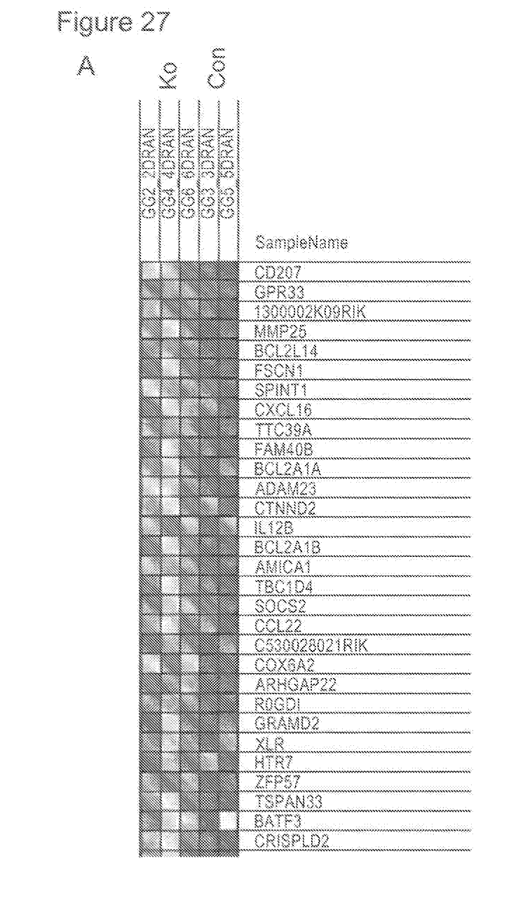

It is possible that PPAR-γ deficiency results in an alteration in the antigen presented in cells in the draining lymph nodes, particularly as myeloid cells are the major producers of CCL17 and CCL22. In this context, an Immgen module for Langerhans' Cells (LC) was enriched in KO LN compared to controls (FIG. 27). Consistent with this idea, published reports show that PPAR-γ can be expressed by LC.

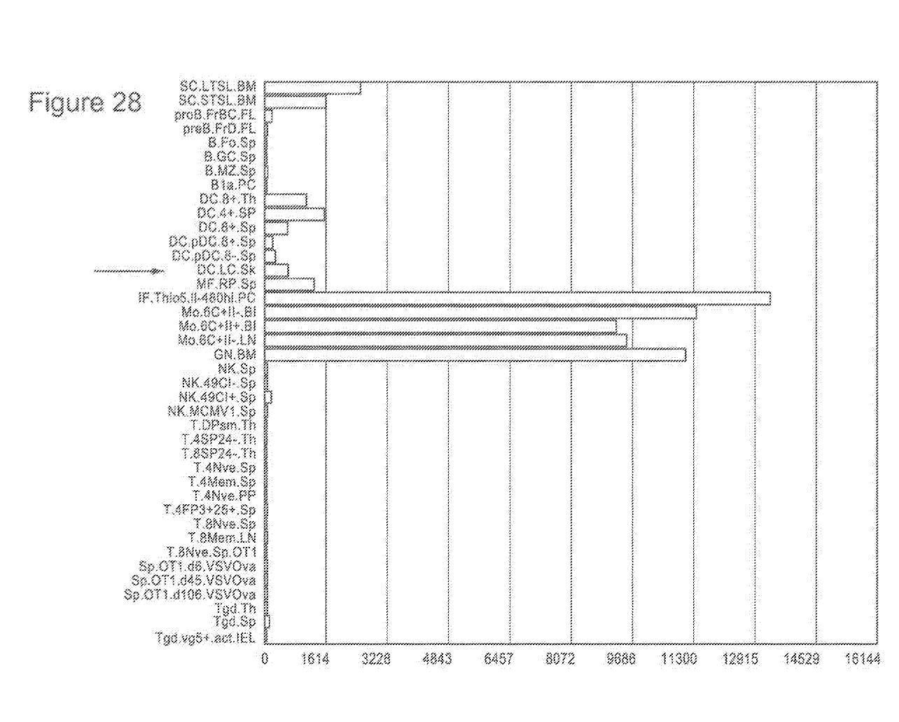

We used the Immgen database to check if Lysozyme M is expressed in LC and thus could be directly impacted by the PPAR-γ deletion. As shown in FIG. 28, LC did express Lysozyme M.

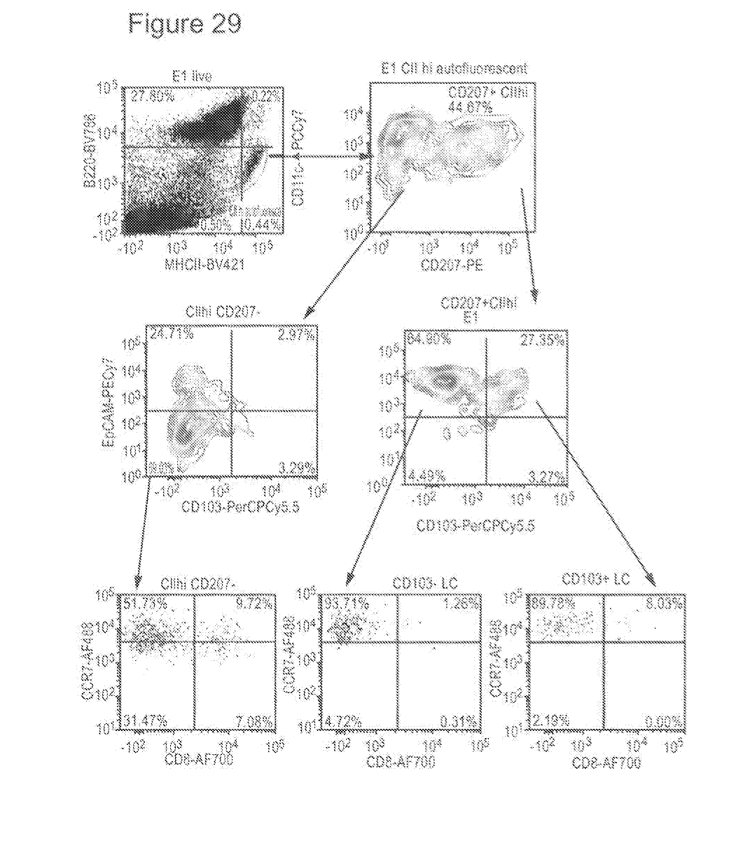

LC travel to the cutaneous lymph nodes upon activation. LC express langerin or CD207. However in recent studies dermal DC have also been shown to express CD207. Further discrimination based on EpCAM and CD103 is possible though there is still some debate over their utility in defining skin dendritic cell subsets [2]. FIG. 29 shows our staining strategy to identify LC and discriminate between LC and dermal langerin expressing DC. We identified LC as CD207+ EpCAM+ cells. We could detect two subsets based on CD103 expression. Further we could detect a CD207− MHCIIhi EpCAM-dendritic cell subtype. All 3 subsets of DC expressed CCR7, suggesting that these are migratory DC. Thus, the CD207− subset might be dermal DC. As expected the LC were negative for CD8 expression.

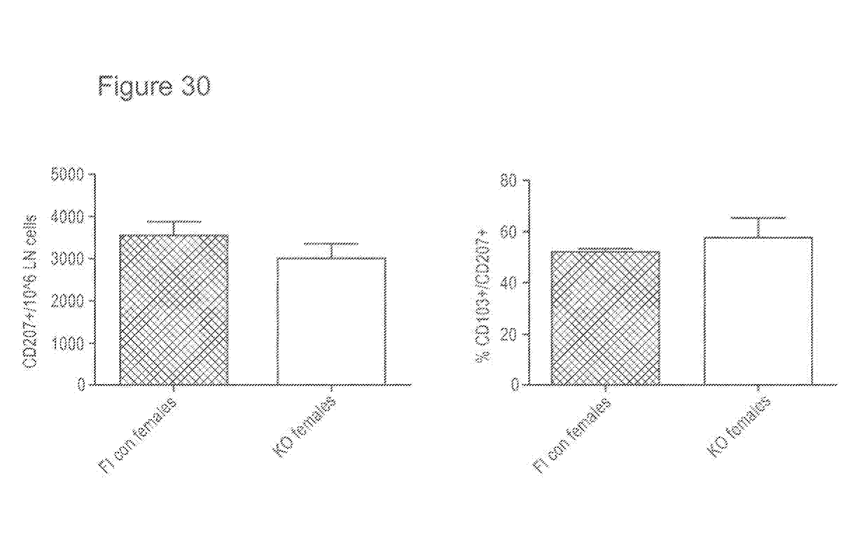

Based on our gating strategy we could not identify a numeric defect in total LC or the relative population of CD103+ LC (FIG. 30). Further studies are required to determine if LC function is altered with PPAR-γ deficiency. We are planning coculture studies of LN APC with various T cell subsets to identify potential functional defects in the KO.

Pharmacological Activation of PPAR-γ Using Rosiglitazone Showed Consistent Gain-of-Function Phenotypes and Identified its Potential as an Immunotherapeutic

Several synthetic agonists of PPAR-γ are available. One of these, Rosiglitazone (Rosi), is well characterized and is clinically approved for the management of diabetes. Given that in our model system, selective loss of PPAR-γ causes increased Treg numbers, we tested if Rosiglitazone treatment in mice treated with GVAX would reduce Treg numbers and improve the CD8:Treg ratio in the LN and the tumor.

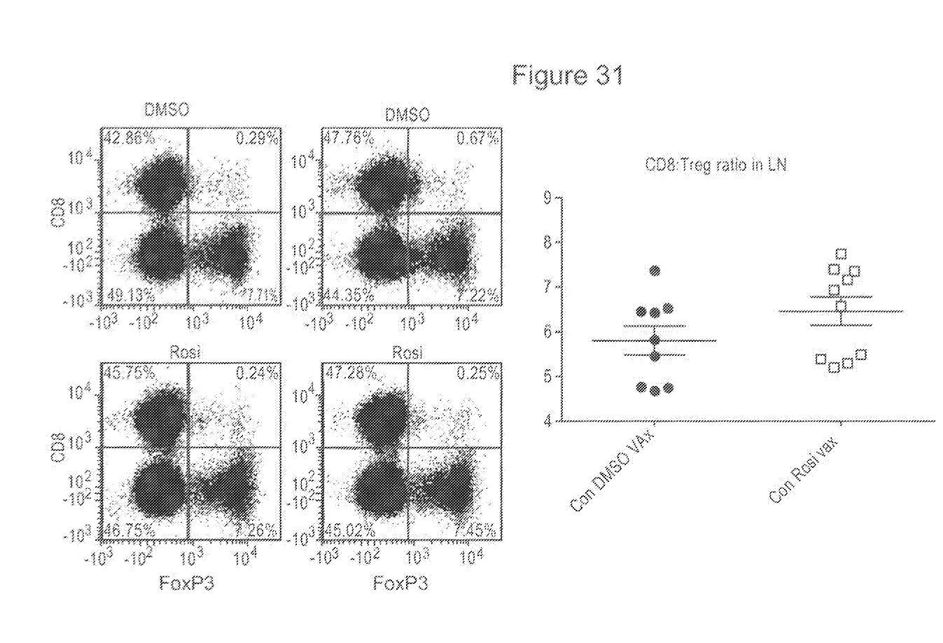

Rosi is given orally to patients. Therefore, we decided to deliver it via drinking water to mice. We started Rosi treatment on the same day as vaccination. To make the Rosi GOF experiments comparable to the genetic LOF, we compared DMSO and Rosi treated LN 6-8 days after vaccination. As shown in FIG. 31, there were no significant differences in CD8 or Treg frequency or in the CD8:Treg ratio in Rosi or DMSO treated GVAX mice. (there appears to be a trend towards an increased CD8/Treg ratio)

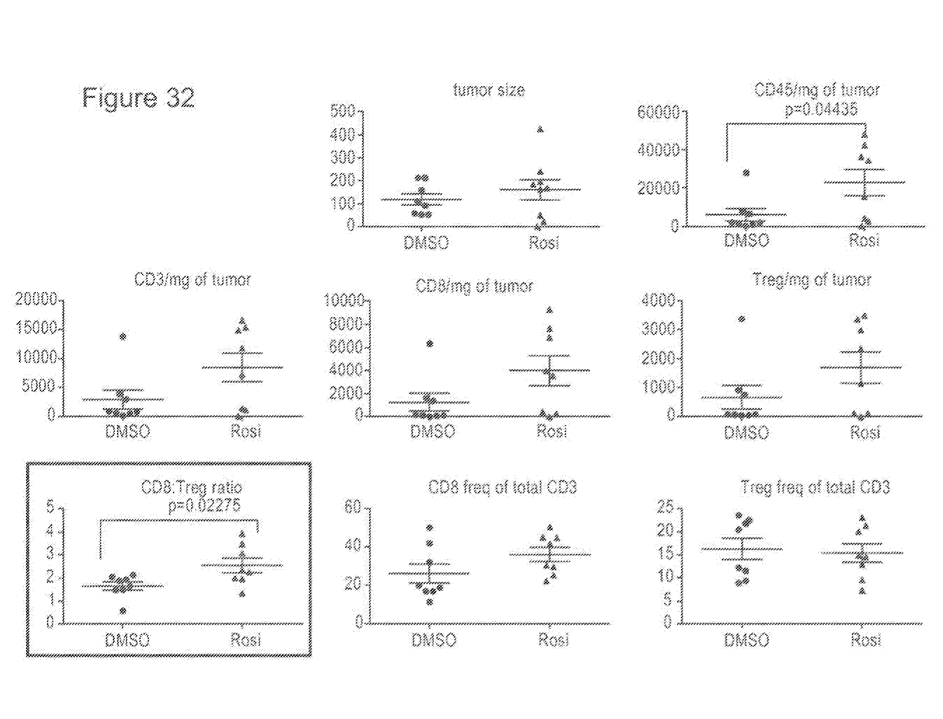

However, Rosi treatment for 12 days showed significant enhancement on the tumor infiltrating lymphocytes (FIG. 32). Strikingly, while KO mice had reduced CD3 infiltration, Rosi treated mice had improved CD3 infiltration and total CD45+ infiltration. Consistent with this, while absolute numbers of CD8 and Treg were higher, Rosi treated mice had higher CD8:Treg ratio. This gain-of-function phenotype is consistent with the genetic loss-of-function of PPAR-γ in the myeloid lineage.

Improved CD8:Treg Ratio with Systemic Delivery of Rosi Requires Myeloid PPAR-γ

It is expected that oral Rosi treatment would impact several cell types. Thus we wanted to address if Rosi mediated improvement in immune infiltrates did require myeloid PPAR-γ. As shown in FIG. 33, CD45+ infiltrate, CD3+ infiltrate as well as CD8:Treg ratio remained unchanged (no statistical significance) with Rosi treatment in the absence of PPAR-γ expression in myeloid cells.

Rosi Improves the Anti-Tumor Response to Combinatorial Treatment with GVAX and CTLA-4 Blockade

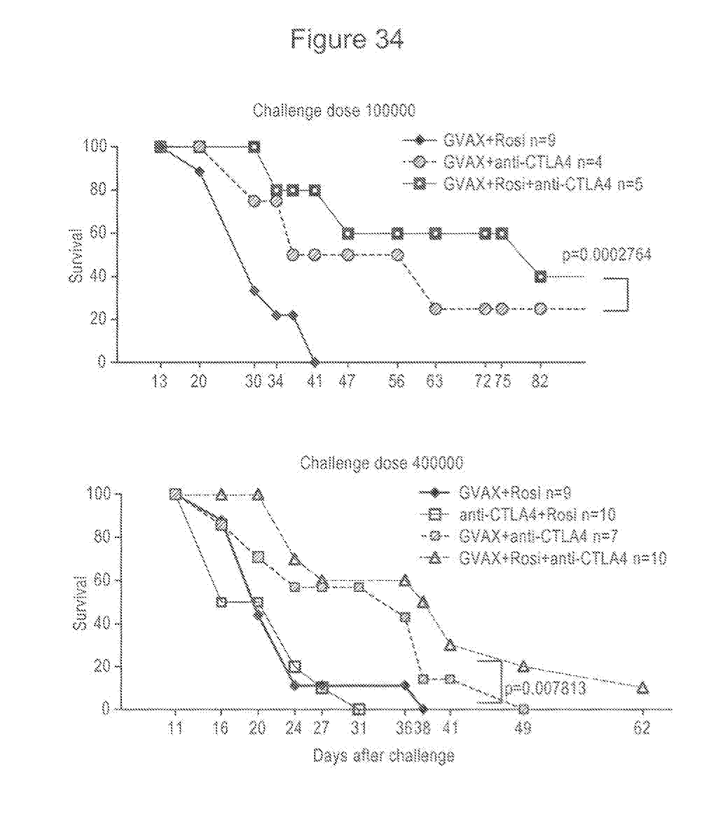

We noticed that Rosi treatment of mice vaccinated with irradiated, GM-CSF secreting B16 cells did not impact the size of the challenge tumor despite having an improved CD8:Treg ration (FIG. 32). Consistent with this result, the combined treatment failed to prolong survival (data not shown). We wondered, however, whether the improvement in the CD8/Treg ratio might result in enhanced efficacy of other combinatorial strategies known to augment vaccination potency. In this context, CTLA-4 antibody blockade is known to improve intratumoral CD8 function and to deplete intra-tumoral Treg in combination with GVAX. CTLA-4 had also emerged as an upregulated gene in KO dLN. Further, T-cells (both effector and regulatory) homing to B16 are known to express CTLA-4. Thus, we tested the effect of Rosi treatment on the response to GVAX+CTLA4. As shown in FIG. 34, Rosi treatment significantly increased survival with GVAX+CTLA4. The benefits of Rosi were observed against two different challenge doses.

### Discussion

We have revealed a previously unidentified function of PPAR-γ in myeloid cells: restraining Treg numbers in response to GM-CSF secreting tumor cell vaccination in mice. This function of PPAR-γ differs from the previously described immunosuppressive effects. However, in our assays, this immunostimulatory role of PPAR-γ is dominant, as GVAX efficacy is reduced in the KO. We have demonstrated that PPAR-γ loss resulted in increased Treg numbers in dLN and tumors, decreased effector to Treg ratios and increased Treg recruiting cytokines in lymph node supernatants. To delineate the contribution of increased Treg to the vaccine defect, we are testing vaccine efficacy in con and KO mice depleted of preexisting Treg using an anti-CD25 antibody.

We were able to demonstrate consistent GOF phenotypes using a synthetic ligand of PPAR-γ, Rosiglitazone. Further, we were able to show that Rosi can potentiate the immune response to GVAX+CTLA-4. These are potentially clinically relevant data as Rosi is an FDA approved small molecule and could be evaluated in patients as a potential immunotherapeutic.

### Example 5: Effect of PPAR-γ Modulation in Studies of GM-CSF Function in Human PBMC

Methods

Culture of Human PBMC with GM-CSF

Human PBMC were obtained by gradient centrifugation of leukapheresis collars from platelet donors. 4×10̂6 cells were plated with 10̂5 K562-WT or K562-GM. Control and GM treated conditions were exposed to 10 uM Rosi or DMSO every 48 hours. On day 4-6 of culture, cells were harvested. Adherent cells were obtained by incubation with 2 mM EDTA at 37° C. Cells were stained for flow cytometry in the presence of 1 mM EDTA. Dead cells were discriminated by using the Live/Dead Fixable dyes from Invitrogen. Antibodies were sourced from BD Biosciences, Biolegend and Ebioscience.

PPAR-γ Modulation

Rosi was obtained from Adipogen as a powder. It was resuspended in DMSO and 10 uM Rosi or equal volume of DMSO was used every 48 hours. T0070907, an antagonist of PPAR-γ, was used at luM added every 48 hours.

CCL17 Measurement

CCL17 levels were measured using ELISA (DY364, R&D Systems).

Results

Human Peripheral Blood Mononuclear Cell Cultures Show Increased Treg Cells on Treatment with GM-CSF which is Counteracted by Myeloid PPAR-γ Agonism

We found that PPAR-γ ligand Rosi can reduce the extent of GM-CSF induced Treg expansion (FIG. 35a, b). The conservation of this pathway between mice and humans is further emphasized by the increase in GM-CSF induced Treg expansion by PPAR-γ antagonist (FIG. 35c). The studies with PPAR-γ antagonist mimic the murine genetic loss-of-function. PPAR-γ modulation was only effective in the presence of GM-CSF and not in cultures with K562-WT.

Rosi Reduces CCL17 Production by Primary Human Monocytes Treated with GM-CSF

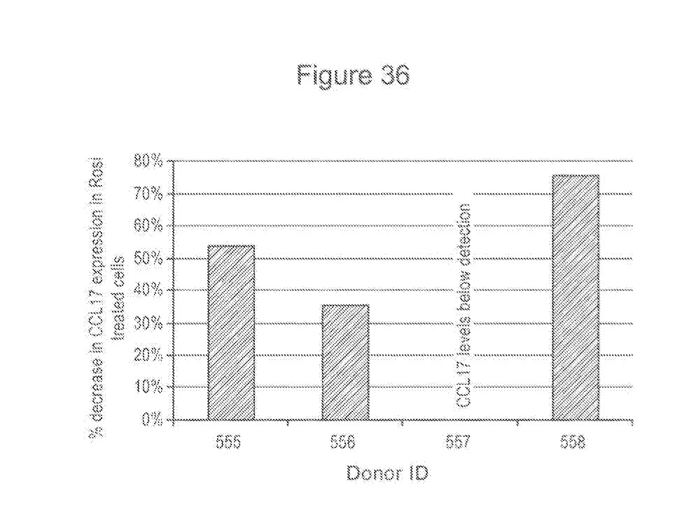

To test if PPAR-γ activation would also reduce the chemokine overexpression that was seen in the murine loss-of-function studies, we cultured CD14+ cells from human PBMC with GM-CSF. In addition, these cultures were treated with DMSO or Rosi. In preliminary data, we find that Rosi treatment reduces CCL17 production by human monocytes treated with GM-CSF (FIG. 36).

Rosi Did not Impact Activation Status of Control or GM Treated Monocytes in the Adherent PBMC

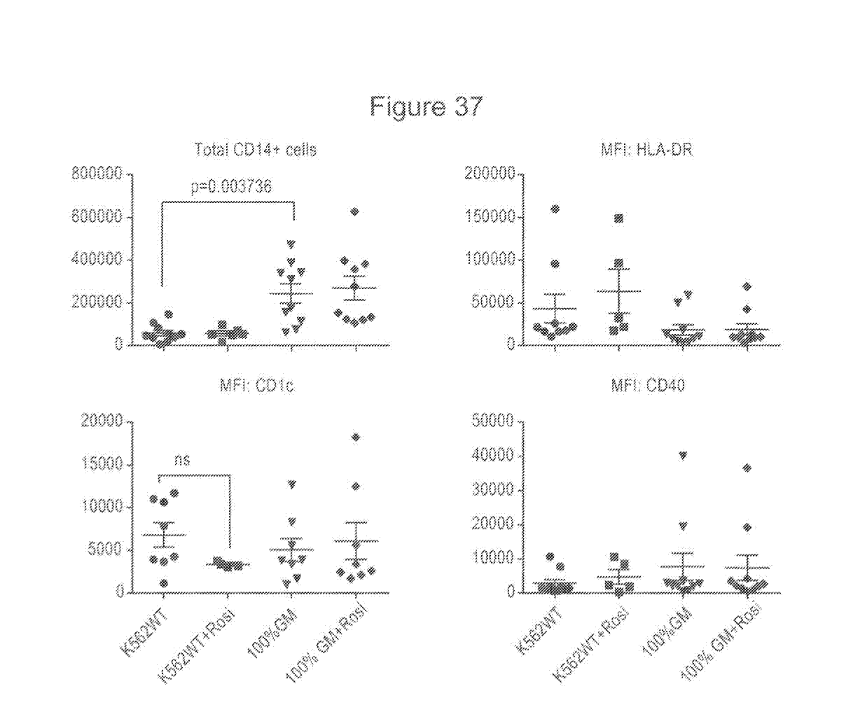

We next evaluated the impact of Rosi treatment on the myeloid cells in culture. Total myeloid cells were calculated based on scatter and CD14 positivity. HLA-DR and CD40 expression was quantified as a measure of activation. Further the adherent cells expressed CD1c suggestive of a dendritic cell phenotype (data not shown). Total myeloid cell number, activation status or expression of CD1c was not affected in Rosi treated control or GM conditions (FIG. 37).

### Discussion

The studies described above show the role of PPAR-γ in restraining GM-CSF induced Treg is conserved in humans. In the PBMC culture, all cells are exposed to Rosi and thus it is possible that we are observing the sum of effects on various cell types. However, it is important to note that Rosi is able to reduce Treg number only in the presence of GM-CSF implying a requirement for myeloid cells. Together with the murine data, our studies have identified a novel and therapeutically important function of PPAR-γ.

1. A method of increasing the efficacy of an active immunotherapy treatment regimen in a subject having a cancer, the method comprising administering a PPAR gamma agonist to the subject receiving the active immunotherapy.

2. The method of claim 1, wherein the active immunotherapy is a non-specific active immunotherapy or a specific active immunotherapy.

3. The method of claim 2, wherein the non-specific active immunotherapy is a cytokine.

4. The method of claim 3, wherein the cytokine is GM-CSF, MCSF or IL-4.

5. The method of claim 4, wherein the GM-CSF is administered via GM-CSF secreting cell or attached to a polymer scaffold.

6. The method of claim 2, wherein the specific active immunotherapy is adoptive T cell therapy or a tumor associated antigen vaccine.

7. The method of claim 6, wherein the T-cell is a chimeric antigen receptor T-cell (CART).

8. The method of claim 1, wherein said subject is further administered an immune check point inhibitor.

9. The method of claim 8, wherein the immune checkpoint inhibitor is an antibody specific for CTLA-4, PD-1, PD-L1, PD-L2 or killer immunoglobulin receptor (KIR).

10. The method of claim 1, wherein said PPAR gamma agonist is a thiazolidinedione.

11. The method of claim 10, wherein the thiazolidinedione is rosiglitazone pioglitazone, troglitazone, netoglitazone, or ciglitazone.

12. The method of claim 1, wherein the cancer is melanoma, non-small cell lung carcinoma (NSCLC), small cell lung cancer (SCLC), bladder cancer or prostate cancer.

13. A method of treating a cancer in a subject comprising administering to said subject a PPAR gamma agonist and an active immunotherapy.

14. The method of claim 13, wherein the non-specific active immunotherapy is a cytokine.

15. The method of claim 14, wherein the cytokine is GM-CSF, MCSF or IL-4.

16. The method of claim 15, wherein the GM-CSF is administered via GM-CSF secreting cell or attached to a polymer scaffold.

17. The method of claim 13, further comprising administering to said subject an immune check point inhibitor.

18. The method of claim 17 wherein the immune checkpoint inhibitor is an antibody specific for CTLA-4, PD-1, PD-L1, PD-L2 or killer immunoglobulin receptor (KIR).

19. The method of claim 13, wherein said PPAR gamma agonist is a thiazolidinedione.

20. The method of claim 19, wherein the thiazolidinedione is rosiglitazone pioglitazone, troglitazone, netoglitazone, or ciglitazone.

21. The method of claim 13, wherein the cancer is melanoma, non-small cell lung carcinoma (NSCLC), small cell lung cancer (SCLC), bladder cancer or prostate cancer.

22. A method of reducing the number of T regulatory cells (Tregs) in a subject in need thereof comprising administering to said subject a PPAR gamma agonist.

23. The method of claim 22, wherein said subject has cancer.

24. The method of claim 22, wherein said subject is receiving an active immunotherapy treatment, an immune checkpoint inhibitor or both.
